(19) United States
(12) Patent Application Publication
(10) Pub. No.: US 2010/0217121 A1
(43) Pub. Date: Aug. 26, 2010

(54) LIQUID INJECTOR, FLUOROSCOPIC IMAGING SYSTEM, AND COMPUTER PROGRAM

(75) Inventor: Shigeru Nemoto, Tokyo (JP)

Correspondence Address: TUROCY & WATSON, LLP, 127 Public Square, 57th Floor, Key Tower, CLEVELAND, OH, 44114, US

(73) Assignee: NEMOTO KYORINDO CO., LTD., Bunkyo-ku, Tokyo (JP)

(21) Appl. No.: 12/680,266

(22) PCT Filed: Sep. 19, 2008
(86) PCT No.: PCT/JP2008/002582

§ 371 Date: Mar. 26, 2010

(30) Foreign Application Priority Data
Sep. 27, 2007 (JP) 2007-251445

(51) Int. Cl. A61B 6/00; G06N 5/02; G05D 7/06; G05D 11/13; G06K 9/00

(52) U.S. Cl. 600/432; 706/54; 700/285; 382/128; 700/282

(57) ABSTRACT

A liquid injector acquires personal condition data originating from a patient, before executing injection of a medical liquid, and a function decision unit decides whether the patient has renal dysfunction based on the acquired personal condition data. Accordingly, such arrangement can be made that in the case where the patient is not decided to have renal dysfunction the liquid injection is executed without announcing an alert, and that in the contrary case the alert is announced and the liquid injection is suspended. Thus, the liquid injector allows easily and surely preventing improper injection of the liquid such as a contrast medium to a patient with renal dysfunction.

### TECHNICAL FIELD

The present invention relates to a liquid injector that injects a medical liquid from a liquid syringe to a patient from whom fluoroscopic image data is to be taken, a fluoroscopic imaging system incorporated with the liquid injector, and a computer program for the liquid injector.

### BACKGROUND ART

Imaging diagnostic apparatuses currently available for picking up a tomographic image, which is fluoroscopic image data of a patient, include a Computed Tomography (CT) scanner, a Magnetic Resonance Imaging (MRI) equipment, a Positron Emission Tomography (PET) equipment, and an ultrasonic diagnostic equipment. Also, medical equipments that pick up a vascular image, which is another fluoroscopic image data of the patient, include a CT angiographic equipment, a Magnetic Resonance Angiographic (MRA) equipment, and so forth.

When one of such equipments is used, the patient often undergoes an injection of a medical liquid, also called a medical fluid, or simply liquid as the case may be, such as a contrast medium or physiological saline, and liquid injectors that automatically execute the injection are currently in practical use. A popular liquid injector retains a liquid syringe loaded with the liquid, and a piston member is press-inserted into the cylinder member of the syringe to thereby inject the liquid into the patient's body.

Although the imaging diagnostic apparatus can work on a stand-alone basis, normally a fluoroscopic imaging system is constituted, including the imaging diagnostic apparatus as part thereof. Such fluoroscopic imaging system includes, for example, a chart management unit, an imaging management unit, an imaging diagnostic apparatus, a data storage unit, and an image viewer.

The chart management unit is generally called a Hospital Information System (HIS), and is utilized to manage so-called electronic charts. The electronic charts each correspond to a patient.

For example, when a patient is to undergo a fluoroscopic image data pickup, the chart management unit makes up imaging order data based on the patient's electronic chart. The imaging order data is generated with respect to each imaging job of picking up the fluoroscopic image data of the patient.

More specifically, the imaging order data includes, for example, imaging job identity (ID) representing exclusive identification data, identification data of the imaging diagnostic apparatus, the patient ID, and date and time of the start and finish of the image pickup.

Such imaging order data is provided to the imaging management unit from the HIS. The imaging management unit is generally called a Radiology Information System (hereinafter, RIS), and serves to store the imaging order data used for picking up a fluoroscopic image data of the patient.

The imaging diagnostic apparatus acquires the imaging order data from the RIS, and executes the imaging job. In other words, the imaging diagnostic apparatus picks up the fluoroscopic image data of the patient according to the imaging order data. The fluoroscopic image data is allocated with at least a part of the imaging order data in the imaging diagnostic apparatus, and then output to the data storage unit.

The data storage unit, generally called a Picture Archive and Communication System (PACS) or alike, stores therein the fluoroscopic image data allocated with the imaging order data.

To the PACS, an image viewer, generally called a viewer, is connected. The image viewer reads out the fluoroscopic image data utilizing, for example, the imaging order data as the retrieval key, and displays that fluoroscopic image data.

Regarding the foregoing fluoroscopic imaging system, various proposals have been made (for example, patented documents 1 and 2).

[Patent document 1] JP-A No. 2001-101320

[Patent document 2] JP-A No. 2005-198808

### DISCLOSURE OF THE INVENTION

The foregoing fluoroscopic imaging system includes the liquid injector that injects the liquid such as a contrast medium from the liquid syringe to the patient whose fluoroscopic image data is to be picked up by the imaging diagnostic apparatus, and hence enables obtaining the fluoroscopic image data of better quality.

However, in the case where the patient has impaired renal function, the injected liquid such as a contrast medium may provoke a physical problem because of insufficient filtration.

Such personal renal dysfunction of the patient is recorded on the chart paper or electronic chart provided that it is known. In the actual site of the liquid injection, accordingly, the renal function is confirmed with the chart before the liquid injection.

It is however troublesome to confirm the renal function of the patient referring to the chart paper or electronic chart at the actual site of the liquid injection. In particular, whereas glomerular filtration rate is typically employed as index of renal function for deciding whether the contrast medium may be injected, generally the glomerular filtration rate is not registered on the chart paper or electronic chart.

Also, even in the case where the renal function is somewhat impaired, the physician may choose to carefully inject the contrast medium manually. Whether the kidney of the patient can effectively filtrate the injected contrast medium depends on the concentration of the contrast medium, injection rate and duration, total injection amount, as well as injection rate and duration, total injection amount, and injection timing of physiological saline to be additionally injected, and so forth.

In other words, in the case where the physician manually injects the contrast medium to the patient with renal dysfunction, the physician comprehensively takes the concentration of the contrast medium, injection rate and duration, total injection amount, as well as injection rate and duration, total injection amount, and injection timing of physiological saline to be additionally injected into consideration, for manually adjusting the injecting operation. However such work is highly complicated and prone to incur erroneous operation.

The present invention has been accomplished in view of the foregoing problem, with an object to provide a liquid injector that allows easily and surely preventing improper injection of a medical liquid such as a contrast medium to a patient with renal dysfunction, a fluoroscopic imaging system that includes such liquid injector, and a computer program for the liquid injector.

According to the present invention, there is provided a liquid injector that injects a medical liquid to a patient whose fluoroscopic image data is to be picked up, comprising a liquid injection mechanism that executes injection of the medical liquid, an injection control unit that controls an action of the liquid injection mechanism according to injection control data to be given, a data input unit that acquires personal condition data originating from the patient before executing the liquid injection, and a function decision unit that decides renal dysfunction of the patient based on the personal condition data acquired.

Thus, with the liquid injector according to the present invention, the injection control unit controls the action of the liquid injection mechanism according to the corresponding injection control data, so as to cause the liquid injection mechanism to inject the liquid to the patient whose fluoroscopic image data is to be picked up. In this process, the data input unit acquires the personal condition data originating from the patient before the liquid injection, and the function decision unit decides whether the patient has renal dysfunction based on the acquired personal condition data. Such arrangement enables, for example, executing the liquid injection without announcing an alert in the case where the patient is not decided to have renal dysfunction, and announcing the alert and suspending the liquid injection in the case where the patient is decided to have renal dysfunction.

In the liquid injector according to the present invention, the data input unit may acquire the personal condition data containing a serum creatinine value, and the function decision unit may decide that the patient has renal dysfunction in the case where the serum creatinine value in the personal condition data deviates from a predetermined acceptable range.

In the liquid injector according to the present invention, the data input unit may acquire the personal condition data containing a glomerular filtration rate, and the function decision unit may decide that the patient has renal dysfunction in the case where the glomerular filtration rate in the acquired personal condition data deviates from a predetermined acceptable range.

In the liquid injector according to the present invention, the data input unit may acquire the personal condition data containing the serum creatinine value, age and body weight, and the function decision unit may calculate an estimated value of the glomerular filtration rate based on the serum creatinine value, age and body weight in the acquired personal condition data, and decide that the patient has renal dysfunction in the case where the estimated value thus acquired deviates from a predetermined acceptable range.

In the liquid injector according to the present invention, the function decision unit may calculate the estimated value of the glomerular filtration rate through a formula of:

(140−age)×body weight/72×serum creatinine value.

In the liquid injector according to the present invention, the data input unit may acquire the personal condition data containing the serum creatinine value, age, body weight, and sex, and the function decision unit may calculate an estimated value of the glomerular filtration rate based on the serum creatinine value, age, body weight, and sex in the acquired personal condition data, and decide that the patient has renal dysfunction in the case where the estimated value thus acquired deviates from a predetermined acceptable range.

In the liquid injector according to the present invention, the function decision unit may calculate the estimated value of the glomerular filtration rate through a formula of:

(140−age)×body weight/72×serum creatinine value

in the where the sex is male, and through a formula of:

(140−age)×body weight/72×serum creatinine value×0.85

in the case where the sex is female.

The liquid injector according to the present invention may further include an alert notification unit that outputs a confirmation alert in the case where the patient is decided to have renal dysfunction.

In the liquid injector according to the present invention, the injection control unit may disable the liquid injection mechanism from working in the case where the patient is decided to have renal dysfunction.

In the liquid injector according to the present invention, the injection control unit may adjust the injecting action of the liquid injection mechanism in the case where the patient is decided to have renal dysfunction.

In the liquid injector according to the present invention, the injection control unit may adjust the injecting action of the liquid injection mechanism according to the acquired serum creatinine value.

In the liquid injector according to the present invention, the injection control unit may adjust the injecting action of the liquid injection mechanism according to the acquired glomerular filtration rate.

In the liquid injector according to the present invention, the injection control unit may adjust the injecting action of the liquid injection mechanism according to the calculated estimated value of the glomerular filtration rate.

In the liquid injector according to the present invention, the injection control unit may adjust at least one of injection rate, injection duration, and a total injection amount provided by the liquid injection mechanism.

In the liquid injector according to the present invention, the liquid injection mechanism injects contrast medium and physiological saline as medical liquid, and the injection control unit may adjust an injection ratio of the contrast medium and the physiological saline.

The liquid injector according to the present invention may further include a liquid acquisition unit that acquires liquid condition data originating from the medical liquid, and the injection control unit may adjust the injecting action according to the liquid condition data in the case where the patient is decided to have renal dysfunction.

In the liquid injector according to the present invention, the liquid injection mechanism may drive a liquid syringe bearing the liquid condition data and loaded with the medical liquid, and the liquid acquisition unit may acquire the liquid condition data from the liquid syringe.

In the liquid injector according to the present invention, the liquid injection mechanism may drive a liquid syringe on which an RFID chip containing the liquid condition data is mounted, and the liquid acquisition unit may acquire the liquid condition data from the RFID chip.

In the liquid injector according to the present invention, the injection control unit may further include a control setting unit that sets at least a part of the acquired liquid condition data in the injection control unit as at least a part of the injection control data.

In the liquid injector according to the present invention, the data input unit may acquire imaging order data of each imaging job containing the personal condition data and managed outside, and the function decision unit may decide whether the patient has renal dysfunction based on the personal condition data in the acquired imaging order data.

The liquid injector according to the present invention may further include a control setting unit that sets at least a part of the acquired imaging order data in the injection control unit as at least a part of the injection control data.

The liquid injector according to the present invention may further include a data output unit that transmits a decision result of renal dysfunction to outside, to thereby store the decision result outside together with the imaging order data.

The liquid injector according to the present invention may further include a history generation unit that generates injection history data containing action history of the liquid injection mechanism based on the injection control data, and a data output unit that transmits the generated injection history data to outside, to thereby store the injection history data outside together with the fluoroscopic image data.

In the liquid injector according to the present invention, the history generation unit may register at least a part of the injection control data in the injection history data.

In the liquid injector according to the present invention, the history generation unit may register the decision result of renal dysfunction in the injection history data.

According to the present invention, there is provided a first fluoroscopic imaging system comprising an external processor that contains imaging order data relevant to each imaging job of picking up fluoroscopic image data from a patient, and the liquid injector according to the present invention that injects a medical liquid to the patient, wherein the external processor contains the imaging order data including personal condition data originating from the patient.

According to the present invention, there is provided a second fluoroscopic imaging system comprising a liquid injector that injects a medical liquid to a patient whose fluoroscopic image data is to be picked up, and a data storage unit that stores therein the fluoroscopic image data picked up, wherein the liquid injector is the one according to the present invention, and the data storage unit also stores injection history data received from the liquid injector, together with the fluoroscopic image data.

According to the present invention, there is provided a computer program for a liquid injector that includes a liquid injection mechanism for injecting a medical liquid to a patient whose fluoroscopic image data is to be picked up, comprising causing the liquid injector to execute an injection control process including controlling an action of the liquid injection mechanism according to injection control data to be set, a data input process including acquiring personal condition data originating from the patient, and a function decision process including deciding whether the patient has renal dysfunction based on the personal condition data acquired.

It is to be noted that each constituent of the present invention has only to be capable of performing its function, and may be constituted in a form of, for example, an exclusive hardware that performs a predetermined function, a data processor in which a predetermined function is incorporated as a computer program, a predetermined function realized in a data processor by a computer program, and an optional combination thereof.

Also, the constituents of the present invention do not necessarily have to be individually independent, but may be configured such that a plurality of constituents constitutes a single member, a constituent is composed of a plurality of members, a constituent is a part of another constituent, a part of a constituent and a part of another constituent overlap, and so forth.

The liquid syringe referred to in the present invention may be a prefilled syringe in which the liquid is loaded and the liquid condition data is recorded by the syringe manufacturer. Otherwise, the liquid syringe may be one delivered from the manufacturer in a form of a refill syringe to the medical site, so that the liquid is loaded and the liquid condition data is recorded at the medical site.

With the liquid injector according to the present invention, the data input unit acquires the personal condition data originating from the patient before the liquid injection, and the function decision unit decides whether the patient has renal dysfunction based on the acquired personal condition data. Such arrangement enables, for example, executing the liquid injection without announcing an alert in the case where the patient is not decided to have renal dysfunction, and announcing the alert and suspending the liquid injection in the case where the patient is decided to have renal dysfunction. Thus, the liquid injector according to the present invention allows easily and surely preventing improper injection of the liquid such as a contrast medium to a patient with renal dysfunction.

### BRIEF DESCRIPTION OF THE DRAWINGS

The above and other objects, features and advantages will become more apparent through a preferred embodiment described hereunder and the following accompanying drawings.

FIG. 1 is a schematic block diagram showing a logical structure of a liquid injector according to an embodiment of the present invention;

FIG. 2 is a schematic block diagram showing a logical structure of a fluoroscopic imaging system;

FIG. 3 is a block diagram showing a physical structure of the fluoroscopic imaging system;

FIG. 4 is a perspective view showing an appearance of a fluoroscopic imaging unit of a CT scanner, an example of an imaging diagnostic apparatus, and an injection head of the liquid injector;

FIG. 5 is a perspective view showing the appearance of the liquid injector;

FIG. 6 is an exploded perpective view showing the injection head of the liquid injector and a liquid syringe;

FIG. 7 is a schematic block diagram showing another logical structure of the liquid injector;

FIG. 8 is a schematic front view showing a screen of the liquid injector, displaying icons of body parts and a condition screen in blank;

FIG. 9 is a schematic front view showing a screen displaying the body part and a region to be imaged that have been selected;

FIG. 10 is a schematic front view showing a screen displaying injection control data;

FIG. 11 is a schematic front view showing a screen displaying a guidance message indicating that acquisition of a patient ID is in process;

FIG. 12 is a schematic front view showing a screen displaying a guidance message indicating an acquisition error of the patient ID;

FIG. 13 is a schematic front view showing a screen displaying an example of the injection condition data;

FIG. 14 is a schematic front view showing a screen displaying another example of the injection condition data;

FIG. 15 is a schematic front view showing a screen displaying liquid condition data;

FIG. 16 is a schematic front view showing a screen indicating the injection control data that has been set;

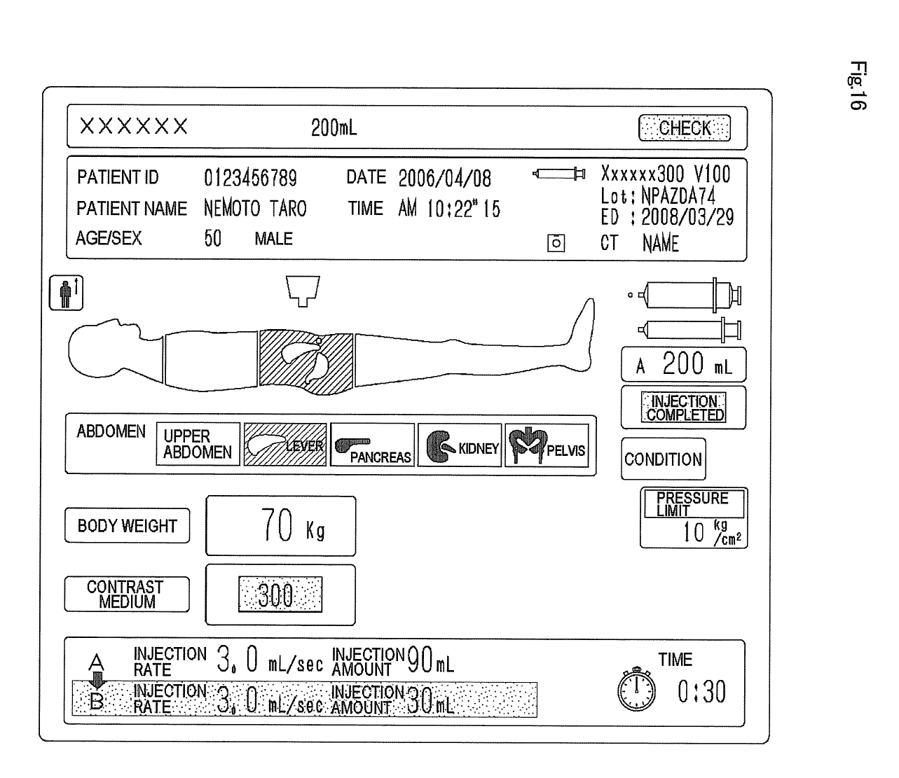

FIG. 17 is a schematic front view showing a screen displaying a guidance message indicating a reference error of the patient ID;

FIG. 18 is a schematic front view showing a screen displaying a time-based graph representing a liquid injection process based on the injection control data manually set;

FIG. 19 is a schematic front view showing a screen displaying a time-based graph representing a liquid injection process based on the liquid condition data and injection condition data that have been automatically set;

FIG. 20 is a schematic front view showing a screen displaying guidance data of an alert that appears when the patient is decided to have renal dysfunction;

FIG. 21 is a flowchart showing a first portion of a process performed by the liquid injector;

FIG. 22 is a flowchart showing a second portion of the process performed by the liquid injector;

FIG. 23 is a flowchart showing a third portion of the process performed by the liquid injector;

FIG. 24 is a schematic time chart showing a processing sequence of the fluoroscopic imaging system.

FIG. 25 is a perspective view showing an appearance of an injection head of a modified liquid injector; and

FIG. 26 is a perspective view showing an appearance of an injection head of another modified liquid injector.

### BEST MODE FOR CARRYING OUT THE INVENTION

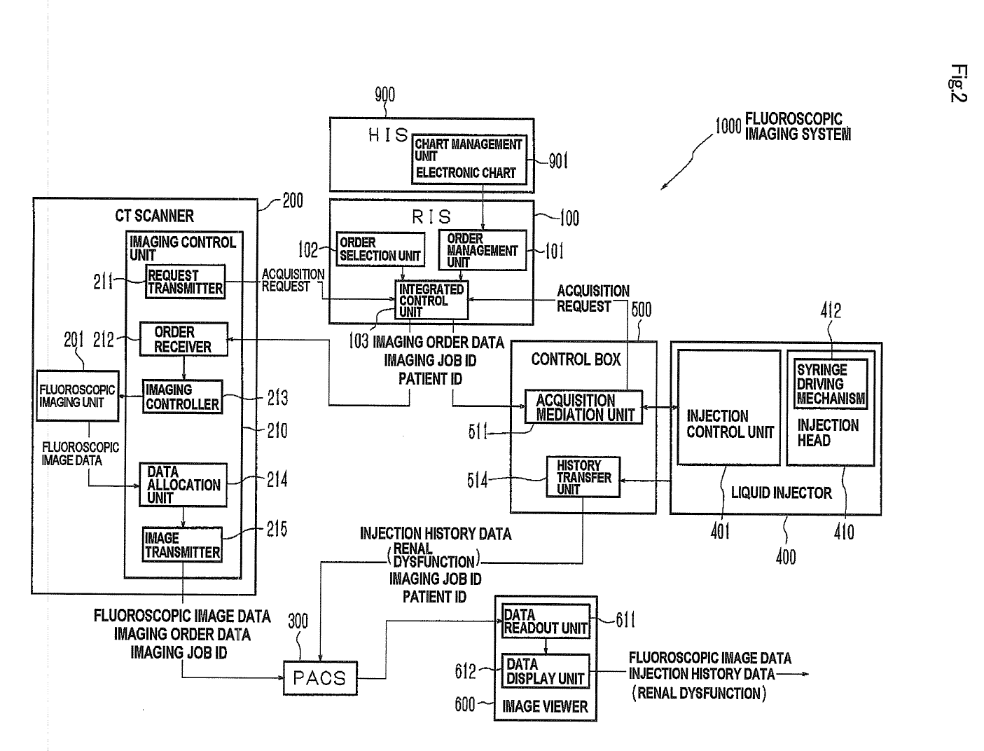

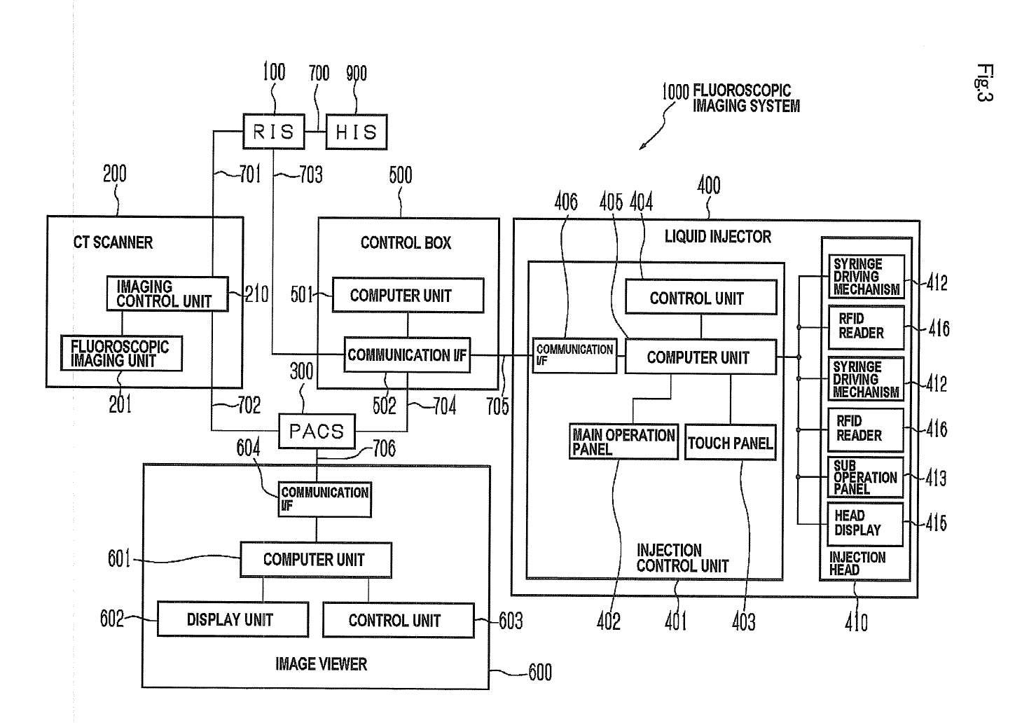

Hereunder, an embodiment of the present invention will be described referring to the drawings. A fluoroscopic imaging system 1000 according to the embodiment of the present invention includes, as shown in FIGS. 2 and 3, a RIS 100 which serves as an imaging management unit, a CT scanner 200 which serves as an imaging diagnostic apparatus, a PACS 300 which serves as a data storage unit, a liquid injector 400, a control box 500 which serves as a data control unit, an image viewer 600, and a HIS 900 which serves as a chart management device.

In the fluoroscopic imaging system 1000 according to this embodiment, the HIS 900 is connected to the RIS 100, through communication network 700 such as a Local Area Network (LAN), as illustrated.

The RIS 100 and the PACS 300 are connected to the CT scanner 200 through the communication networks 701, 702. Likewise, the control box 500 is connected to the RIS 100, the PACS 300, and the liquid injector 400 through communication networks 703 to 705. To the PACS 300, the image viewer 600 is connected through a communication network 706.

The fluoroscopic imaging system 1000 according to this embodiment is based on what is known as Digital Imaging and Communications in Medicine (DICOM). Accordingly, the respective units 100 to 600, and 900 of the fluoroscopic imaging system 1000 mutually communicate according to DICOM specification.

In the fluoroscopic imaging system 1000 according to this embodiment, one each of the CT scanner 200, the PACS 300, the liquid injector 400, the control box 500, and the HIS 900 are provided, and all the combinations of these units are on a one-to-one basis.

The HIS 900 according to this embodiment is constituted of a known computer unit, in which an exclusive computer program is installed. In the HIS 900, units such as a chart management unit 901 are logically realized as the functions thereof, to be activated when the computer unit executes the corresponding processes according to the computer program.

The chart management unit 901 corresponds to a storage area of a hard disc drive (hereinafter, HDD) recognized according to the computer program, in which the electronic chart of each patient is stored.

The electronic chart includes text data such as a chart ID and the patient ID of each individual patient which are exclusive identification data, personal data such the name of the patient, and chart data related to the disease of the patient.

The chart data of the electronic chart contains personal condition data related to overall treatment process including the body weight, sex, age, serum creatinine value, and so on of the patient. Here, the liquid syringe 800 is assumed to be of a prefilled type in this embodiment, and hence the liquid ID corresponds to the product ID of the liquid syringe 800.

The RIS 100 according to this embodiment is also constituted of a known computer unit, and units such as an order management unit 101, an order selection unit 102, and an integrated control unit 103 are logically realized as the functions thereof, to be activated when the computer unit executes the corresponding processes according to the exclusive computer program installed therein.

The order management unit 101 corresponds to a storage area of a HDD, and serves to manage the imaging order data used for picking up the fluoroscopic image data of the patient, with the exclusive identification data. The imaging order data is generated based on the electronic chart acquired from the HIS 900.

The imaging order data thus generated includes text data such as an imaging job ID which is the exclusive identification data, types of the job such as CT scanning or MR imaging, the patient ID and data of the electronic chart, the identification data of the CT scanner 200, body part or region to be imaged, proper types including the type of liquid such as a contrast medium relevant to the imaging job, the proper ID including the liquid ID appropriate for the imaging job, and so forth.

Since the imaging order data contains the data in the electronic chart, the personal condition data in the chart data is also contained. Accordingly, in the imaging order data body weight, sex, age, serum creatinine value, and so on of the patient are also registered.

The order selection unit 102 corresponds to a function assigned to a central processing unit (hereinafter, CPU), of executing a predetermined process according to an input through a keyboard, and selects one from a plurality of imaging order data according to the input by the operator.

The integrated control unit 103 corresponds to a function assigned to the CPU, of transmitting and receiving various data through a communication interface (I/F), and returns the selected one of the imaging order data according to a acquisition request received from the CT scanner 200 or the control box 500.

The CT scanner 200 according to this embodiment includes, as shown in FIG. 3, a fluoroscopic imaging unit 201 which is the image-pickup execution mechanism, and an imaging control unit 210. The fluoroscopic imaging unit 201 shoots the fluoroscopic image data of the patient. The imaging control unit 210 controls the action of the fluoroscopic imaging unit 201.

To be more detailed, the imaging control unit 210 is constituted of a computer unit, in which an exclusive computer program is installed. In the imaging control unit 210, a request transmitter 211, an order receiver 212, an imaging controller 213, a data allocation unit 214, and an image transmitter 215 are logically realized as the functions thereof, when the computer unit executes the corresponding process according to the computer program.

The request transmitter 211 corresponds to a function assigned to the CPU, of transmitting and receiving various data through the communication interface (I/F) according to the input through the keyboard, and transmits the acquisition request for the imaging order data to the RIS 100 according to the input by the operator. The order receiver 212 receives the imaging order data returned from the RIS 100.

The imaging controller 213 controls the action of the fluoroscopic imaging unit 201 according to the imaging order data received. The data allocation unit 214 allocates the imaging order data to the fluoroscopic image data picked up by the fluoroscopic imaging unit 201.

The image transmitter 215 transmits the fluoroscopic image data allocated with the imaging order data to the PACS 300. Here, the fluoroscopic image data thus generated is composed of, for example, bit map data of the tomographic image.

The PACS 300 according to this embodiment is constituted of a database server, in which also an exclusive computer program is installed. The PACS 300 receives the fluoroscopic image data allocated with the imaging order data from the CT scanner 200, and stores the received data.

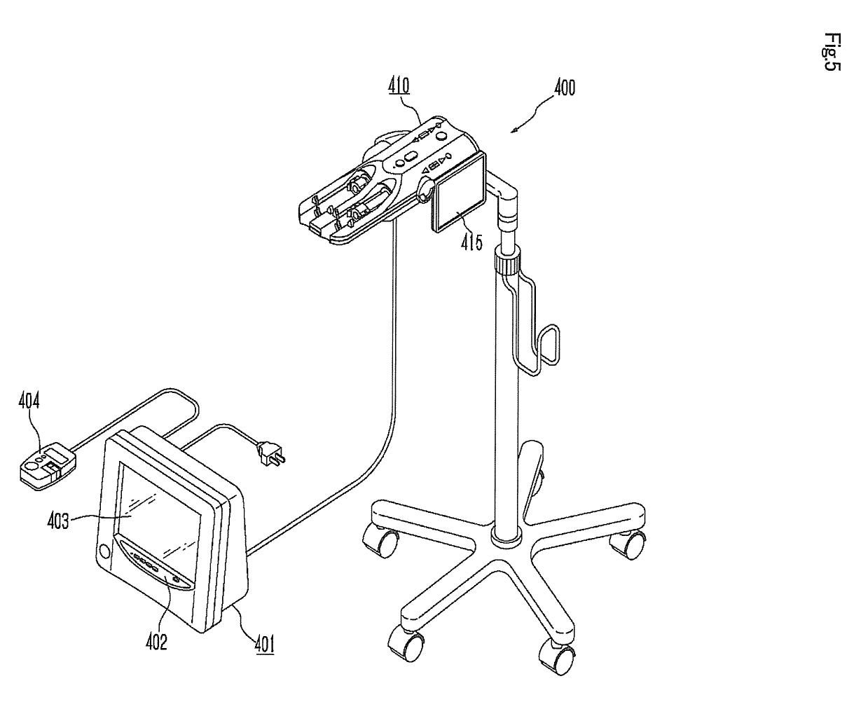

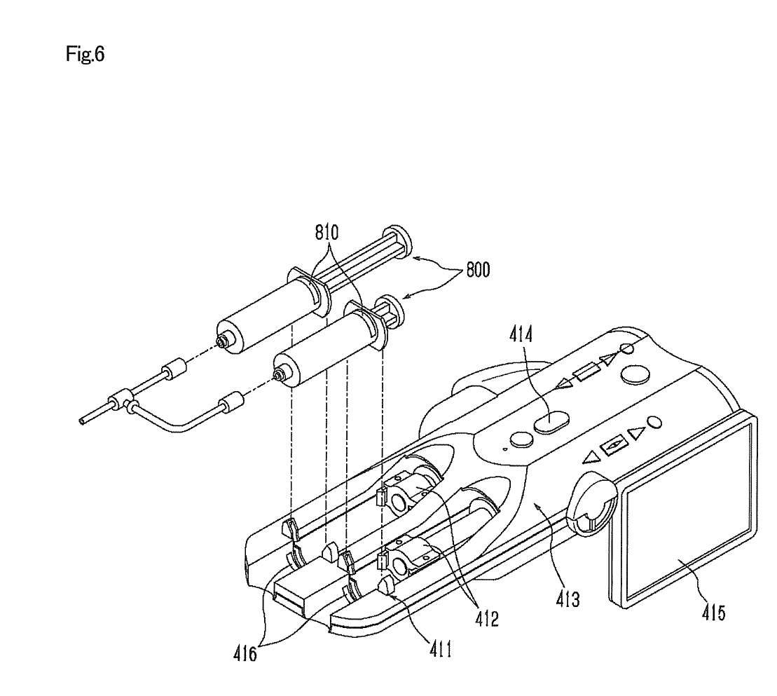

The liquid injector 400 according to this embodiment includes, as shown in FIG. 5, an injection control unit 401 and an injection head 410. The injection control unit 401 controls the action of the injection head 410. The injection head 410 drives a liquid syringe 800, also called a fluid syringe or medical syringe, removably attached thereto as shown in FIG. 6, to thereby inject a liquid into the patient.

To be more detailed, the injection control unit 401 includes, as shown in FIGS. 3 and 5, a main operation panel 402, a touch panel 403, a controller 404, a computer unit 405, and a communication I/F 406.

The injection head 410 includes a syringe holding mechanism 411 constituting a part of the liquid injection mechanism for retaining the liquid syringe 800, a syringe driving mechanism 412 serving as the liquid injection mechanism that drives the liquid syringe 800, a sub operation unit 413 used to input an action instruction to the syringe driving mechanism 412, a head display 415 serving as the data display device that outputs various data for display, and so forth.

The sub operation unit 413 includes a final confirmation switch 414 which will be subsequently described. The head display 415 is directly fixed to a rear lateral portion of the injection head 410, and located close to the syringe holding mechanism 411 and the syringe driving mechanism 412.

Here, various types of syringes may be employed as the liquid syringe 800 according to this embodiment, including one with an RFID chip 810 installed thereon at a predetermined position. On the injection head 410, an RFID reader 416, which serves as a liquid acquisition unit, is attached at such a position that enables making wireless communication with the RFID chip 810 only when the liquid syringe 800 is properly retained by the syringe holding mechanism 411.

The RFID chip 810 of the liquid syringe 800 contains at least liquid condition data regarding the liquid, registered therein. To be more detailed, the liquid syringe 800 is of what is known as a prefilled type shipped with the liquid loaded in advance, and hence the liquid condition data is registered in the RFID chip 810 prior to the shipment.

The liquid condition data include, the data related to the loaded liquid, for example, the proper job type such as CT scanning or MR imaging, the type of liquid such as a contrast medium, the liquid ID including the product ID of the prefilled syringe, various data of the contrast medium such as ingredients, chemical classification, viscosity, and expiry date, as well as various data of the liquid syringe 800 such as capacity, cylinder withstand pressure, cylinder bore, piston stroke, lot number, and price.

The liquid ID of the liquid condition data is registered based on the chemical classifications, ingredients and chemical structure of the liquid, and is not associated with the syringe capacity and the like. For example, in the case where the products of a company A and a company B are available as heart contrast medium for CT scanning, if the chemical classifications, such as whether water-soluble or oil-based, ionic or anionic, monomer type or dimer type are different, the product IDs become different, although the type of the liquid specified as “heart contrast medium for CT scanning” is the same.

Further, although the type of the liquid and chemical classifications are the same, if the ingredients are different the product IDs become different, and even though the type of the liquid, chemical classifications and ingredients are the same, if the chemical structure of even a single ingredient is different, the product IDs become different.

On the other hand, in the case where an identical liquid is loaded in the prefilled liquid syringes of 200 ml and 500 ml in capacity, the product ID of the liquid is the same, though the liquid syringes are different as products by the capacity.

To the computer unit 405 of the liquid injector 400, the respective units cited above are connected. The computer unit 405 integrally controls the computer program, in which the respective units connected to the computer unit 405 are implemented.

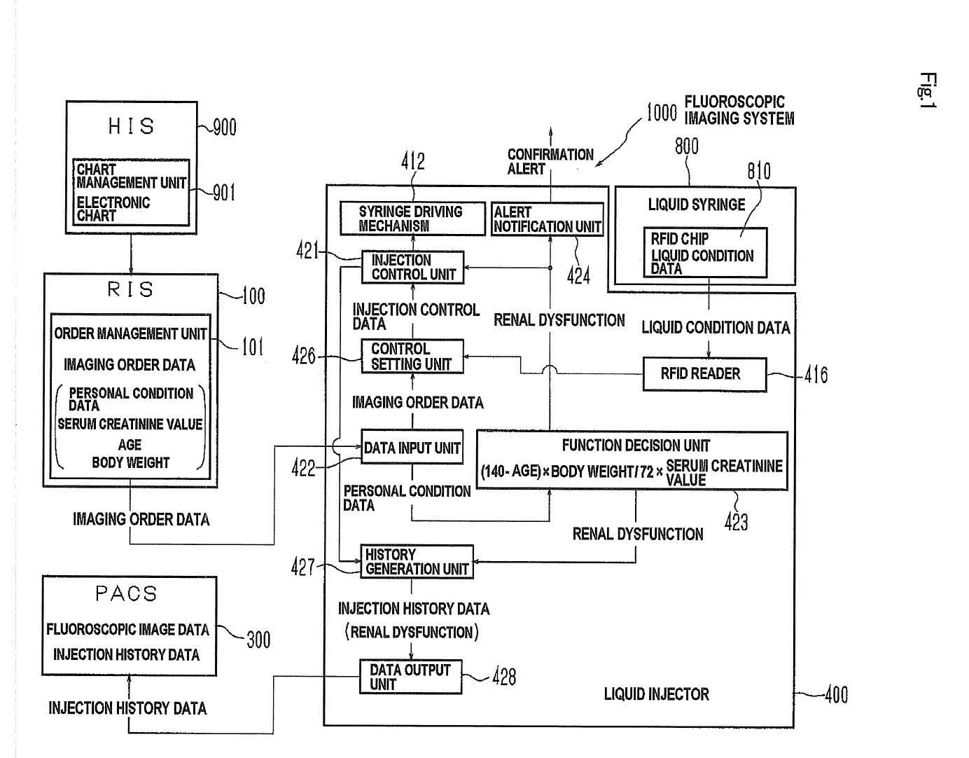

Accordingly, in the liquid injector 400 the following units are logically realized as the functions thereof, as shown in FIG. 1, namely an injection control unit 421 that controls the action of the syringe driving mechanism 412 according to the injection control data to be set, a data input unit 422 that acquires the personal condition data of the patient before executing the liquid injection, a function decision unit 423 that decides whether the patient has renal dysfunction based on the acquired personal condition data, and so on.

To be more detailed, the data input unit 422 acquires the imaging order data of each imaging job containing the personal condition data and managed by the RIS 100, thereby acquiring the personal condition data containing the serum creatinine value, age, and body weight.

The function decision unit 423 calculates an estimated value of the glomerular filtration rate based on the serum creatinine value, age, and body weight in the acquired personal condition data, and decides that the patient has renal dysfunction in the case where the estimated value thus calculated deviates from a predetermined acceptable range.

In this process, the function decision unit 423 utilizes the following formula for calculating the estimated value of the glomerular filtration rate:

(140−age)×body weight/72×serum creatinine value

The injection control unit 421 disables the syringe driving mechanism 412 from working in the case where the patient is decided to have renal dysfunction. The liquid injector 400 also includes an alert notification unit 424 that announces a confirmation alert in the case where the patient is decided to have renal dysfunction. Here, the injection control unit 421 cancels the decision on renal dysfunction in response to a predetermined inputting operation.

More particularly, the injection control unit 421 of the liquid injector 400 corresponds to the function assigned to the computer unit 405, of controlling the action of the syringe driving mechanism 412 according to the computer program.

The data input unit 422 corresponds to the function assigned to the computer unit 405, of making data communication with the RIS 100 and the PACS 300 through the communication I/F 406, according to the computer program. The function decision unit 423 corresponds to the function assigned to the computer unit 405, of executing a predetermined data processing according to the computer program.

The alert notification unit 424 corresponds to the function assigned to the computer unit 405, of controlling the image display on the touch panel 403 and the head display 415, according to the computer program.

The liquid injector 400 also includes a control setting unit 426 that sets at least a part of the acquired liquid condition data in the injection control unit 421 as at least a part of the injection control data, and sets at least a part of the acquired imaging order data in the injection control unit 421 as at least a part of the injection control data.

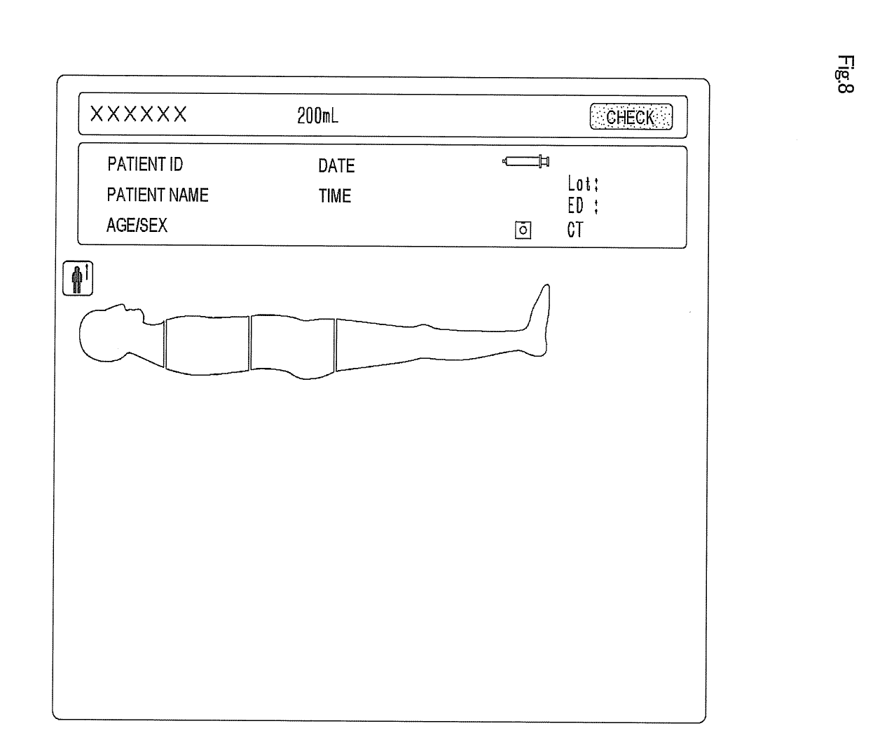

To be more detailed, on the touch panel 403 of the injection control unit 401, an operating icon for inputting the acquisition request, including a profile of a human body and an icon of “i”, is displayed for example in a left upper region of the initial screen for inputting the injection control data, as shown in FIG. 8.

By inputting the acquisition request through manipulating the operating icon, a part of the imaging order data is acquired as the injection condition data, through the control box 500. Then out of the injection condition data, the patient ID and the region to be imaged are set in the injection control unit 421 as at least a part of the injection control data.

The injection control data includes, for example, protocol data in which a moving stroke and pressure of the syringe driving mechanism 412 are specified for different time points, by a predetermined command.

Here, whereas the liquid injector 400 according to this embodiment is capable of automatically setting the injection control data utilizing the foregoing units 421 to 423, manual operation may be executed for introducing new injection control data and editing the setting of the injection control data.

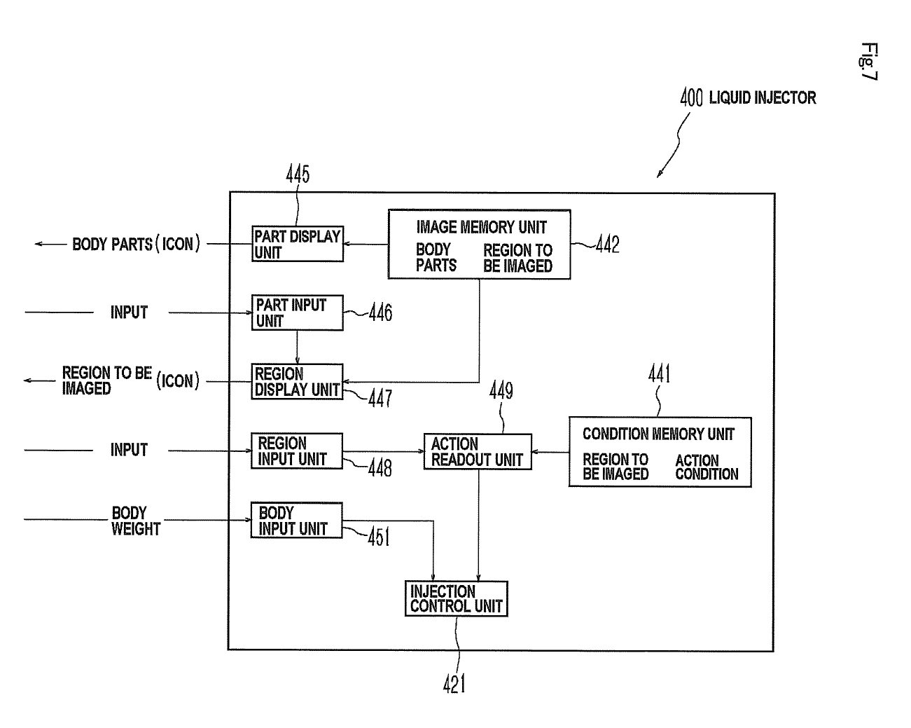

Further, as shown in FIG. 7, in the liquid injector 400 units such as a condition memory unit 441, an image memory unit 442, a part display unit 445, a part input unit 446, a region display unit 447, a region input unit 448, an action readout unit 449, and a body input unit 451 are logically realized as the functions thereof.

The image memory unit 442 of the liquid injector 400 contains the memory of a plurality of human body parts and a multitude of regions to be imaged, in association therebetween. The part display unit 445 displays the icon of the plurality of body parts stored in the image memory unit 442, in a layout corresponding to a human body shape.

The part input unit 446 accepts an input for selecting one of the plurality of body parts displayed on the part display unit 445, as an input of one of the injection condition data. The region display unit 447 outputs and displays an icon of at least a region to be imaged, according to the body part selected through the part input unit 446. The region input unit 448 accepts an input for selecting the region to be imaged displayed on the region display unit 447, as one of the injection condition data.

More specifically, in the liquid injector 400 the plurality of body parts includes the head, chest, abdomen, and leg, and the icons representing each of them are registered in the computer unit 405.

When a predetermined operation is executed with the liquid injector 400, the icons of the head, chest, abdomen, and leg, are displayed in a layout corresponding to a human body shape, in an upper portion of the touch panel 403, as shown in FIG. 8.

Further, icons representing the brain, chin, and neck are registered as a plurality of regions to be imaged, in association with the icon of the head, which is one of the body parts displayed. Likewise, icons of the heart and lung are registered in association with the icon of the chest; icons of the stomach, lever, and so forth in association with the icon of the abdomen; and icons of the upper leg and lower leg in association with the icon of the leg.

Then once one of the icons of the human body shape representing the plurality of body parts displayed on the touch panel 403 is manually touched, an icon representing the scanner mechanism is output and displayed above the selected icon only, and only the manually operated icon is lit up while all the remaining icons are turned off (not shown).

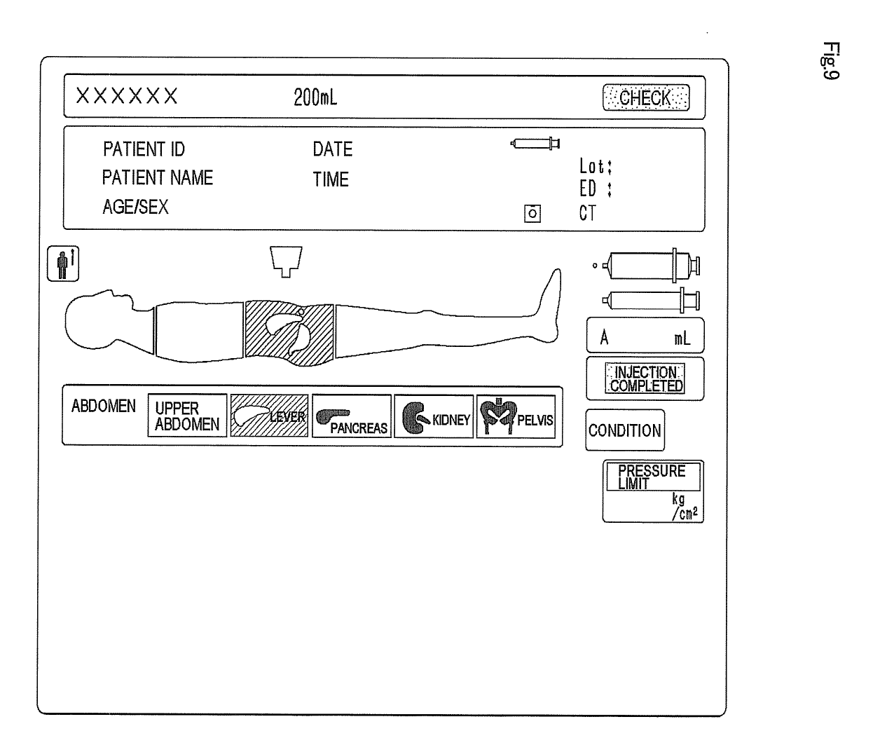

At the same time, below the selected icon, the icons of the plurality of corresponding regions to be imaged are output and displayed. Then when one of the icons representing the plurality of regions to be imaged is manually touched, only the selected icon is lit up and the others are turned out, as shown in FIG. 9.

The condition memory unit 441 stores working condition data of the syringe driving mechanism 412, with respect to each of the multitude of regions to be imaged of the human body. The working condition data is specified, for example, in terms of a total injection amount of a contrast medium to each region to be imaged of the human body.

The action readout unit 449 reads out the working condition data corresponding to the region to be imaged selected through the region input unit 448, from the condition memory unit 441, and sets the data in the injection control unit 421 as a part of the injection control data.

The body input unit 451 accepts an input of body weight, as physical information of the patient who is to undergo the fluoroscopic image data pickup, and sets the body weight in the injection control unit 421 as a part of the injection control data.

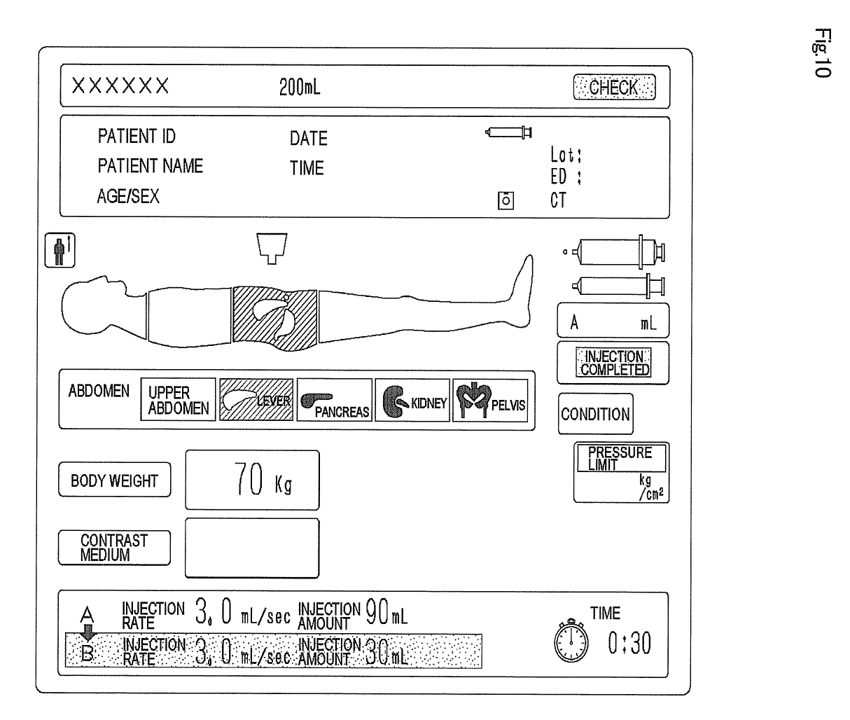

To be more detailed, once the operating icon of “condition” is manually touched after manual selection of the region to be imaged by touching the relevant icon as above, the screen becomes ready to accept an input of body weight, injection amount, injection time and so on. Then upon inputting the value representing the body weight, such value is displayed, and set as a part of the injection control data as shown in FIG. 10.

The liquid injector 400 also includes a history generation unit 427 that generates injection history data containing a history of the action of the syringe driving mechanism 412 taken based on the injection control data, and a data output unit 428 that transmits the generated injection history data to outside, to thereby store the injection history data outside together with the fluoroscopic image data. Here, the history generation unit 427 also registers in the injection history data at least a part of the injection control data, and the decision on renal dysfunction.

Such injection history data is composed, for example, of image data of a time-based graph in which one of the horizontal axis and the vertical axis represents the time elapsed and the other the injection rate, on which the injection control data, the patient ID, the injection job ID and so on are written in text data.

The control box 500 according to this embodiment includes, as shown in FIG. 3, a computer unit 501 in which an exclusive computer program is installed, and a communication I/F 502.

In the control box 500 also, the computer unit 501 executes various processes according to the computer program. Accordingly, units such as an acquisition mediation unit 511, a history transfer unit 514, and so on are logically realized in the control box 500 as the function thereof.

The acquisition mediation unit 511 acquires the imaging order data from the RIS 100 according to the acquisition request received from the liquid injector 400, and returns a part of the acquired imaging order data to the liquid injector 400, as a part of the injection control data. The history transfer unit 514 receives the injection history data from the liquid injector 400, and transfers it to the PACS 300.

Accordingly, the PACS 300 according to this embodiment does not only stores the fluoroscopic image data received from the CT scanner 200, but also stores the injection history data received from the control box 500, as described above.

As already stated, the fluoroscopic image data is allocated with the imaging order data, and the imaging job ID of that imaging order data is allocated to the injection history data. Accordingly, the imaging order data and the injection history data are stored in the PACS 300, mutually associated via the imaging job ID.

The image viewer 600 according to this embodiment also includes a computer unit in which an exclusive computer program is installed. The image viewer 600 includes, as shown in FIG. 3, a computer unit 601, a display unit 602, a controller 603, a communication I/F 604.

The image viewer 600 includes, as shown in FIG. 2, a data readout unit 611 and a data display unit 612, which are realized when the computer unit 601 executes the corresponding process according to the computer program.

The data readout unit 611 corresponds to a function assigned to the computer unit 601, of making access to the PACS 300 through the communication I/F 604 according to the computer program and the data input to the controller 603, and reads out the fluoroscopic image data and the injection history data associated via the imaging job ID, from the PACS 300.

The data display unit 612 corresponds to the function assigned to the computer unit 601, of causing the display unit 602 to display the data received through the communication I/F 604, and displays the fluoroscopic image data and the injection history data that have been read out.

It is to be noted that the foregoing computer program of the RIS 100 is described as software for causing the RIS 100 to, for example, store the imaging order data containing the imaging job ID, the patient ID, the injection condition data and so forth, select one from a plurality of imaging order data according to an input by an operator, return the selected imaging order data according to the acquisition request from the CT scanner 200 or the control box 500, transfer the liquid condition data received from the control box 500 to the HIS 900, and so forth.

The computer program of the CT scanner 200 is described as software for causing the imaging control unit 210 to, for example, transmit the acquisition request for the imaging order data to the RIS 100 according to an input by the operator, receive the imaging order data returned from the RIS 100, control the action of the fluoroscopic imaging unit 201 according to the imaging order data that has been received, allocate the fluoroscopic image data picked up by the fluoroscopic imaging unit 201 with the imaging order data, and transmit the fluoroscopic image data allocated with the imaging order data to the PACS 300.

Also, the computer program of the liquid injector 400 is described as software for causing the computer unit 405 to, for example, acquire the liquid condition data from the RFID chip 810 of the liquid syringe 800 by means of the RFID reader 416, set at least a part of the acquired liquid condition data and at least a part of the acquired imaging order data as at least a part of the injection control data, acquire the imaging order data of each imaging job managed by the RIS 100, control the action of the syringe driving mechanism 412 according to the injection control data to be set, acquire the serum creatinine value, age and body weight from the acquired imaging order data as the personal condition data, calculate the estimated value of the glomerular filtration rate based on the serum creatinine value, age and body weight in the acquired personal condition data with the formula of:

(140−age)×body weight/72×serum creatinine value,

decide that the patient has renal dysfunction in the case where the calculated estimated value deviates from the predetermined acceptable range, announce the confirmation alert in the case where the patient is decided to have renal dysfunction, disable the syringe driving mechanism 412 from working in the case where the patient is decided to have renal dysfunction, cancel the decision on renal dysfunction in response to the predetermined inputting operation, generate the injection history data containing the action history of the syringe driving mechanism 412 according to the injection control data, and transmit the generated injection history data and the decision on renal dysfunction to the PACS 300, to thereby store therein the injection history data together with the fluoroscopic image data.

The computer program of the control box 500 is described as software for causing the computer unit 501 to, for example, acquire the imaging order data from the RIS 100 in response to the acquisition request from the liquid injector 400, return the patient ID and so on of the acquired imaging order data to the liquid injector 400, receive the injection history data and liquid condition data from the liquid injector 400, and transfer the received injection history data to the PACS 300 and transfer the liquid condition data to the RIS 100.

The computer program of the PACS 300 is described as software for causing the PACS 300 to, for example, receive the fluoroscopic image data allocated with the imaging order data from the CT scanner 200 and store that data, and receive from the control box 500 the injection history data allocated with the imaging job ID of the imaging order data and the decision on renal dysfunction, and store the injection history data.

The computer program of the image viewer 600 is described as software for causing the computer unit 601 to, for example, read out from the PACS 300 the fluoroscopic image data, the injection history data, and the decision on renal dysfunction mutually associated via the imaging job ID, and display the fluoroscopic image data, the injection history data, and the decision on renal dysfunction that have been read out.

Hereunder, a procedure of picking up the fluoroscopic image data of the patient with the fluoroscopic imaging system 1000 thus configured according to this embodiment will be sequentially described. To start with, the operator registers in advance the imaging order data in the RIS 100.

The imaging order data contains the text data including the imaging job ID, the identification data of the CT scanner 200, date and time of the start and finish of the image pickup, and the region to be imaged. The imaging order data is normally made up based on the electronic chart of each patient.

The operator who makes up the imaging order data manually operates the RIS 100 so as to acquire the electronic chart from the HIS 900. Accordingly, the ID, name and body weight of the patient are also registered in the imaging order data.

Here, as stated earlier the electronic chart also contains the body weight, sex, age, and the serum creatinine value of the patient as the personal condition data. Accordingly, such personal condition data is also registered in the imaging order data.

However, the imaging order data contains various data that is necessary for the imaging job with the CT scanner 200. Accordingly, the imaging order data does not contain such data that permits identifying the injection job performed by the liquid injector 400.

When the imaging job is executed with such imaging order data registered in the RIS 100, the operator may manually operate the RIS 100, to thereby select one of the imaging order data corresponding to the imaging job.

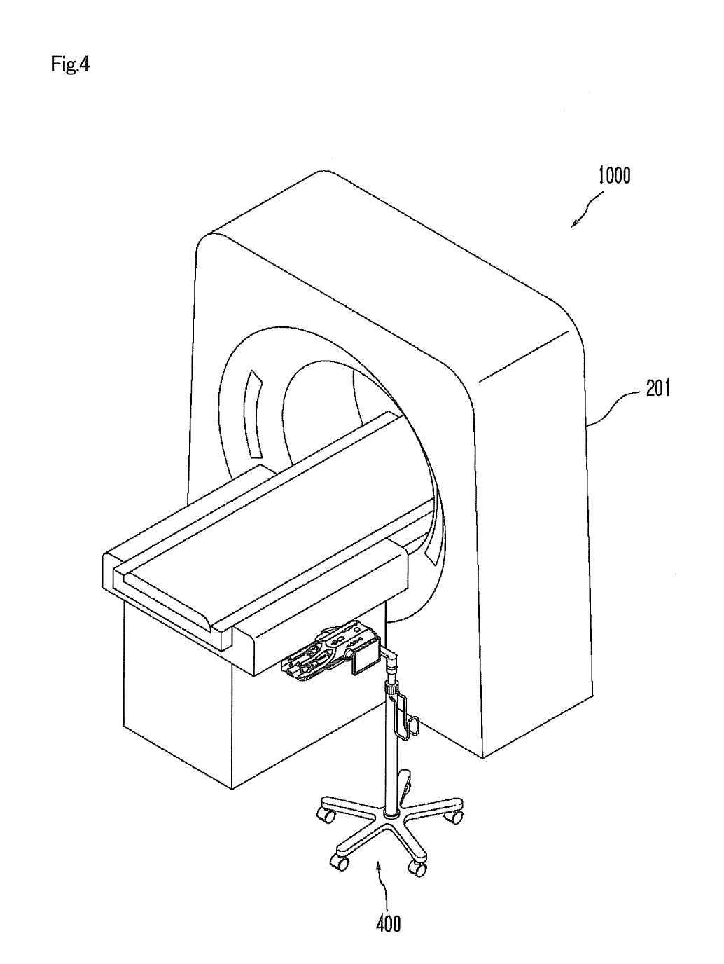

Meanwhile at the actual site of the imaging job, the liquid injector 400 is located close to the fluoroscopic imaging unit 201 of the CT scanner 200, as shown in FIG. 4. Then the liquid syringe 800 is connected to the patient (not shown) in the fluoroscopic imaging unit 201 through an extension tube, and the liquid syringe 800 is loaded onto the injection head 410 of the liquid injector 400.

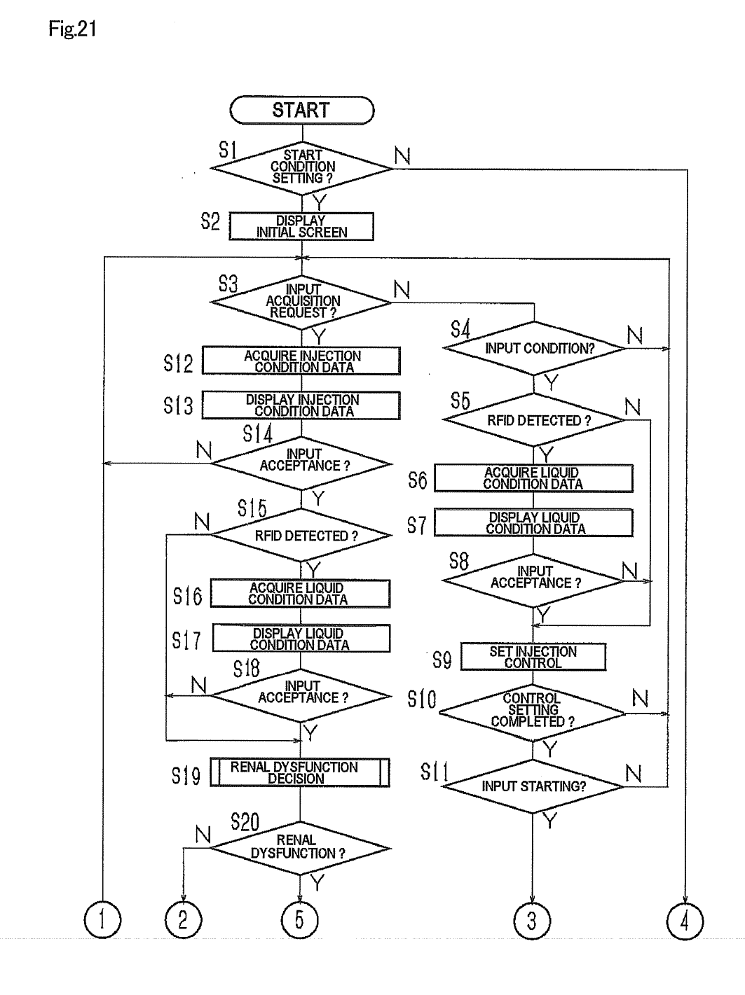

Once the operator activates the liquid injector 400 for example by an inputting action through the main operation panel 402 of the injection control unit 401 as shown in FIG. 21 (step S1), the icons representing a plurality of body parts are displayed on the touch panel 403, as shown in FIG. 8 (step S2).

The liquid injector 400 according to this embodiment does not permit the action control on the syringe driving mechanism 412 based on the injection control data, in the initial stage where the injection control data is not set. While the liquid injector 400 accepts manual setting of the entirety of injection control data at the stage where the initial screen is displayed as above, it is also possible to automatically set a part of the injection control data based on the imaging order data.

In the case of manual setting, the operator presses with a finger one of the plurality of icons representing the body parts displayed on the touch panel 403. Then only the selected part of the icon is lit up while all the remaining parts are turned off, and an icon of the scanner mechanism is displayed above the selected icon of the body part.

At the same time, below the selected part, icons of a plurality of regions to be imaged corresponding to the selected body part are read out and displayed in the selection screen. When the operator inputs one of the icons by a press of a finger, only the selected icon is lit up and the others are turned out, as shown in FIG. 9.

Once the region to be imaged is thus selected, in the liquid injector 400 the action condition data corresponding to the region to be imaged is read out and set as the injection control data. Also, as shown in FIG. 10, the body weight of the patient, injection rate, total injection amount, injection time and so on are input as the injection control data to the main operation panel 402, by the operator (step S4).

At this stage, the liquid injector 400 according to this embodiment also confirms with the RFID reader 416 whether the RFID chip 810 is mounted in the liquid syringe 800 (step S5).

In the case where the RFID chip 810 is mounted in the liquid syringe 800, the RFID reader 416 acquires the liquid condition data (step S6). The liquid condition data includes, as already stated, various data on the loaded liquid such as the product name and expiry, and various data on the liquid syringe 800 such as the capacity and lot number.

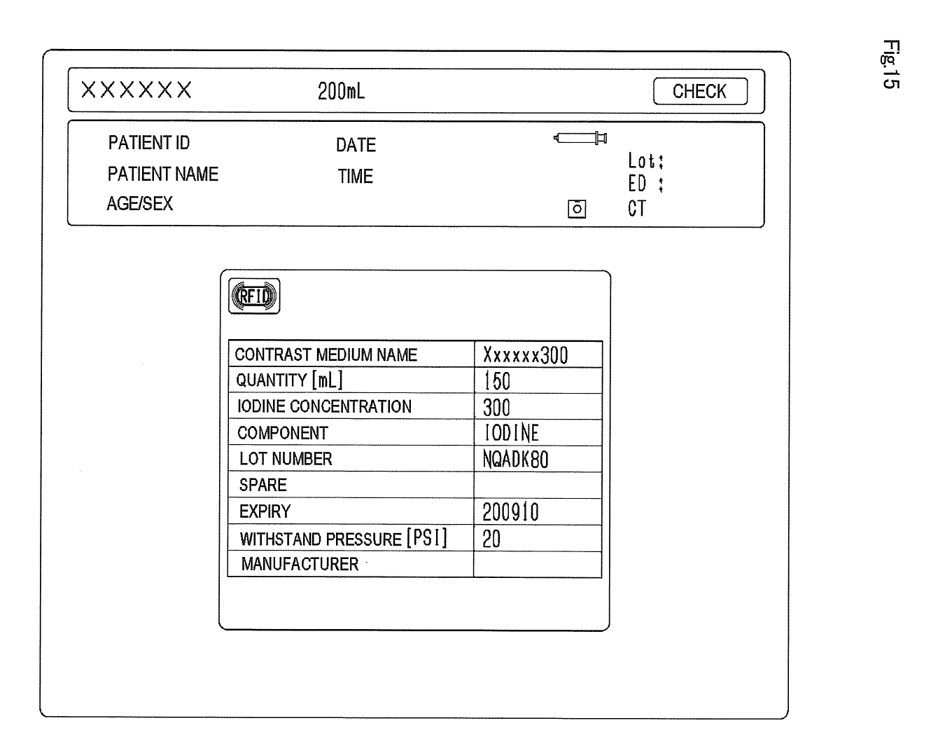

A part of the liquid condition data thus acquired is output for display on the touch panel 403 of the injection control unit 401 and the head display 415 of the injection head 410, as shown in FIG. 15 (step S7).

At this stage, a predetermined “RFID” logo mark appears on the touch panel 403 and the head display 415, indicating that the liquid condition data being displayed has been acquired from the RFID chip 810 in the liquid syringe 800.

Then the operator confirms the liquid condition data displayed as above and inputs the injection control data (step S8). Once the setting of the injection control data has been thus completed (steps S9, S10), the liquid injector 400 becomes ready to accept the input of the instruction to start the injection.

Inputting the starting instruction through the touch panel 403 (step S11) activates the syringe driving mechanism 412 according to the injection control data set as above, so that the contrast medium and physiological saline is properly injected to the patient.

The fluoroscopic imaging system 1000 according to this embodiment also allows, however, automatically setting the injection control data in the liquid injector 400, in addition to the foregoing manual setting. More specifically, the liquid injector 400 according to this embodiment also displays the operating icon of “acquisition request” in an upper left region on the initial screen of the injection job, as shown in FIG. 8.

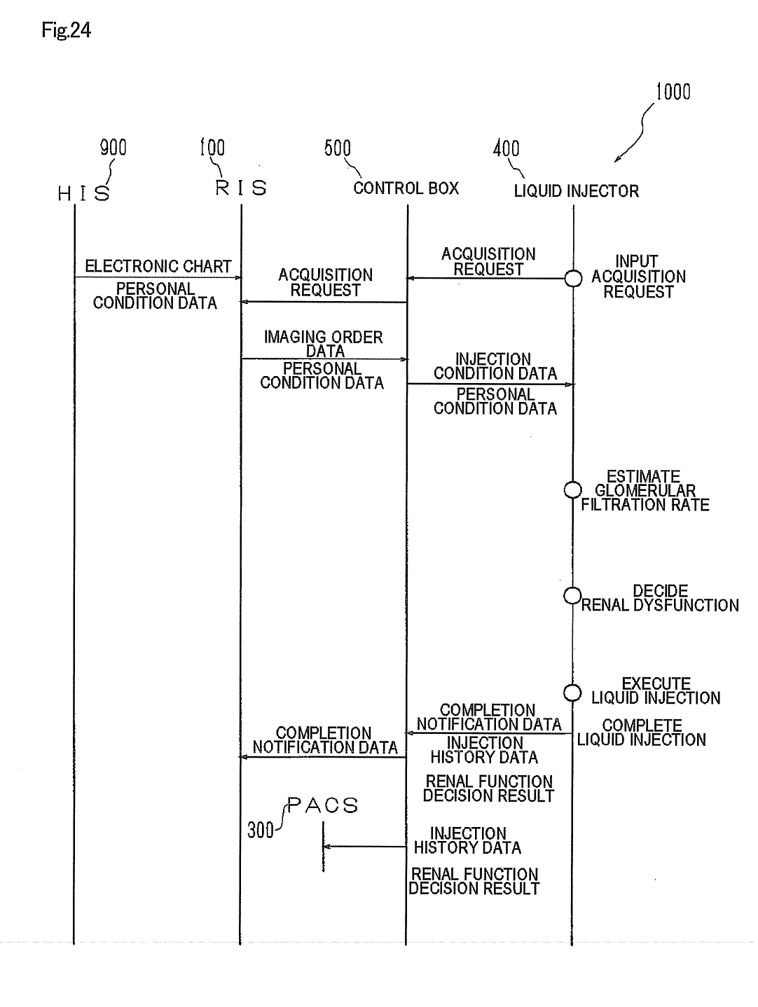

Once the operating icon of “acquisition request” is manually operated (step S3), the acquisition request is transmitted to the control box 500 as shown in FIG. 24. The control box 500 transfers the acquisition request received from the liquid injector 400, to the RIS 100.

The RIS 100 then returns the one of the imaging order data selected as above to the control box 500. The control box 500 returns, upon receipt of the imaging order data from the RIS 100, a part of the imaging order data to the liquid injector 400 as at least a part of the injection condition data.

More specifically, as already stated, the imaging order data includes the imaging job ID, the identification data of the CT scanner 200, the date and time of the start and finish of the image pickup, the patient ID, the body part or region to be imaged, and the personal condition data.

The control box 500 extracts the imaging job ID, the patient ID, the body part or region to be imaged, the personal condition data, and so on out of the acquired imaging order data, and returns such data to the liquid injector 400 as the injection condition data.

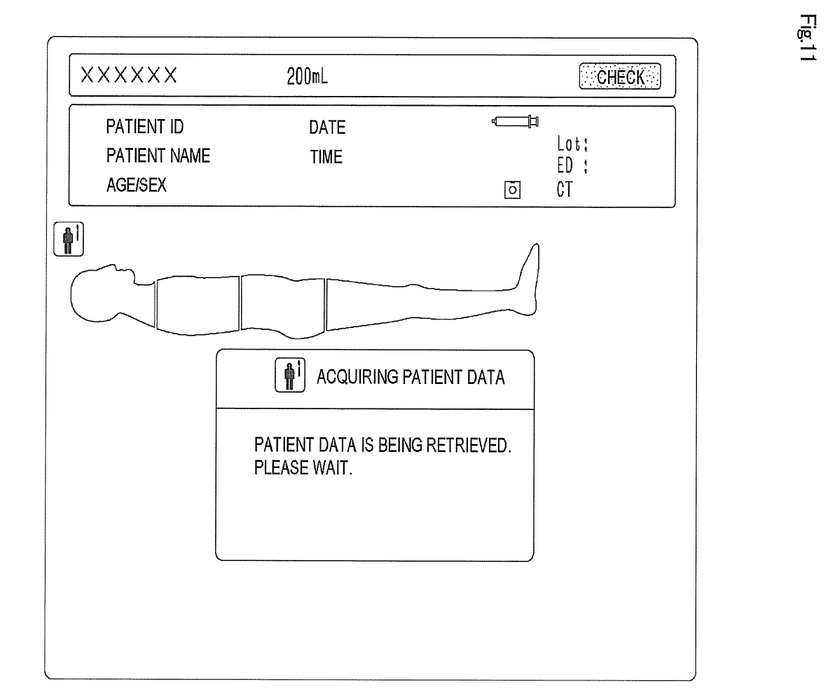

During such communication, the liquid injector 400 displays the guidance data indicating that the communication is being made, on the touch panel 403 and the head display 415, as shown in FIG. 11. Therefore, the operator can confirm at real time that the liquid injector 400 is executing the communication.

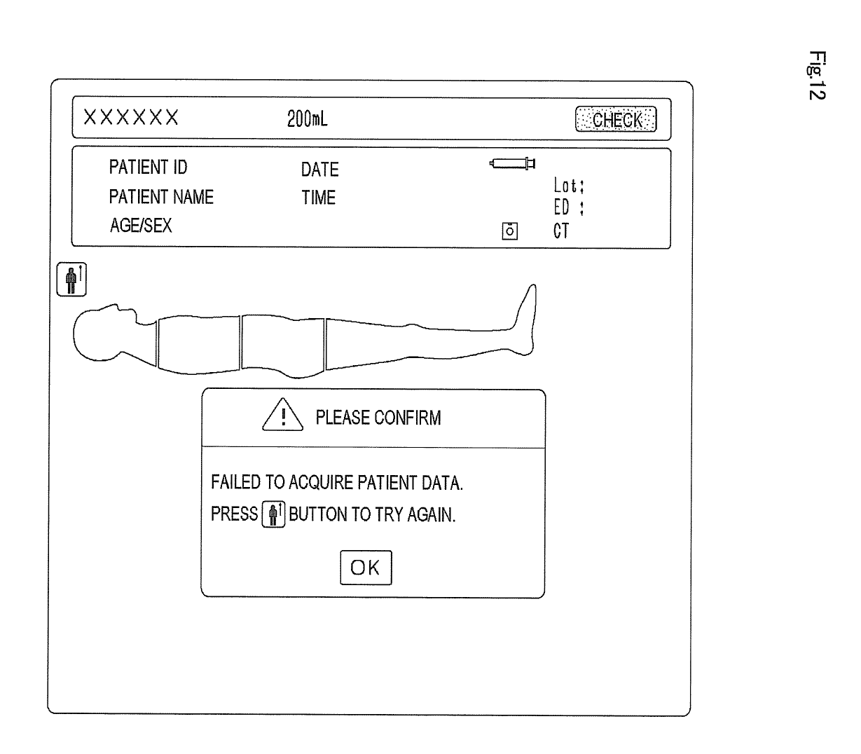

Also, in the case where the injection condition data cannot be acquired because of a communication error or the like, guidance data indicating the failure in acquiring the data is displayed on the touch panel 403 and the head display 415, as shown in FIG. 12. This enables the operator to immediately recognize the failure in data acquisition, and to take another step.

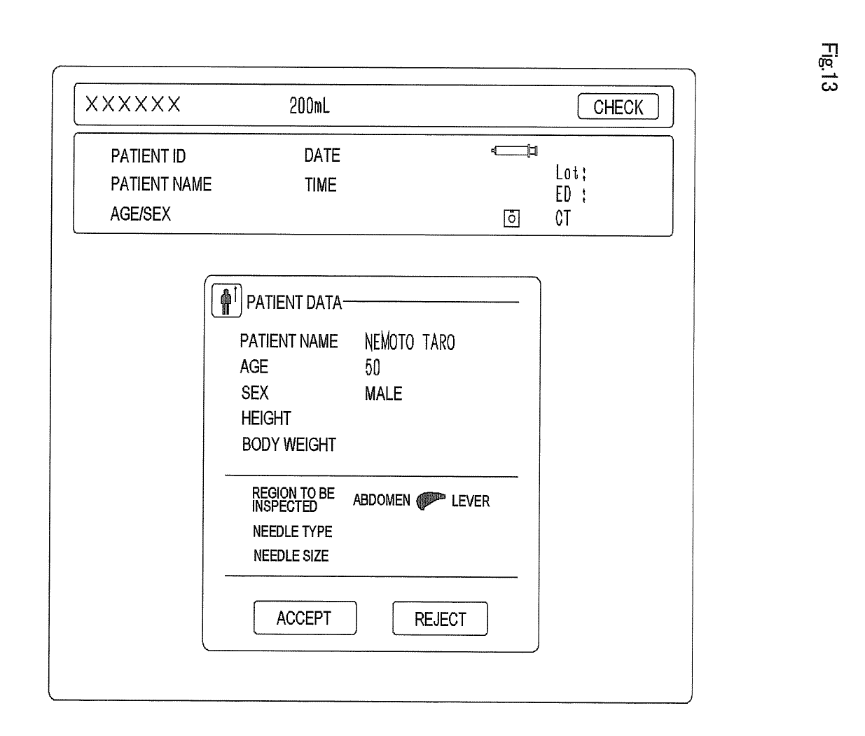

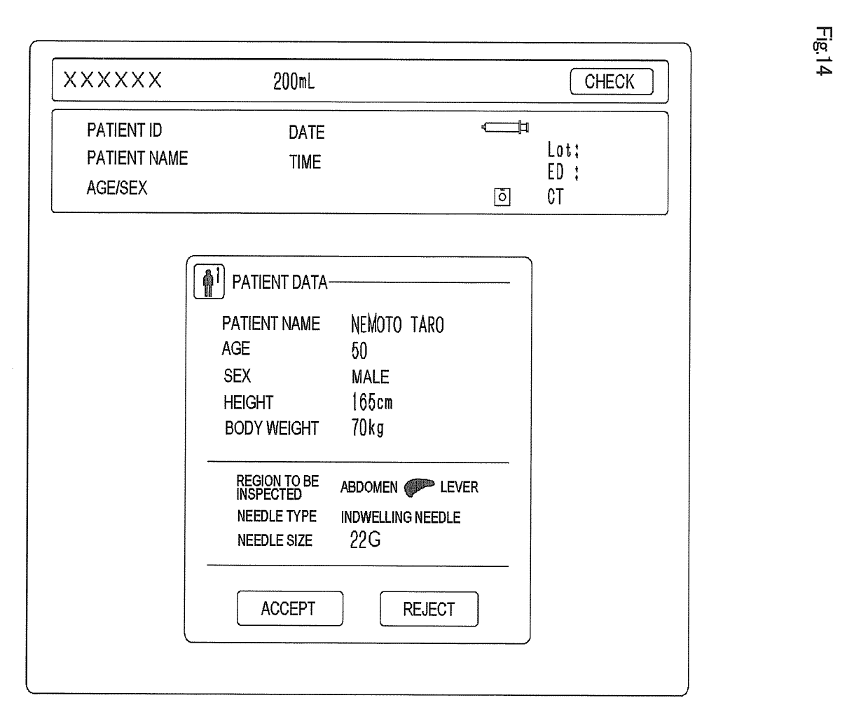

In the liquid injector 400, the injection condition data acquired from the control box 500 in response to the acquisition request (step S12) is displayed on the touch panel 403 and the head display 415, as shown in FIGS. 13 and 14 (step S13).

At the same time, the name and sex of the patient are also displayed as the injection condition data, based on which the operator can check the accordance between the injection condition data and the actual patient. Also, the touch panel 403 and the head display 415 display an operating icon for instructing whether to use the injection condition data as the injection control data, together with the injection condition data displayed as above.

In the case of setting the injection condition data as the injection control data, the operator touches the operating icon indicating “accept”. Then the liquid injector 400 confirms, upon detecting such input (step S14), whether the RFID chip 810 is mounted in the liquid syringe 800, through the RFID reader 416 (step S15).

In the case where the RFID chip 810 is mounted on the liquid syringe 800, the liquid condition data is acquired through the RFID reader 416 (step S16). The liquid condition data contains, as stated earlier, various data on the loaded liquid and various data on the liquid syringe 800.

Here, the data on the liquid includes the proper job type such as CT scanning or MR imaging, the type of liquid such as a contrast medium, the liquid ID including the product ID of the prefilled syringe, the ingredients and chemical classification of the contrast medium, and so forth.

Then a part of the liquid condition data thus acquired is displayed on the touch panel 403 of the injection control unit 401 and the head display 415 of the injection head 410, as shown in FIG. 15 (step S17).

At this stage, a predetermined logomark of “RFID” is displayed on the touch panel 403 and the head display 415, to thereby indicate that the liquid condition data being displayed has been acquired from the RFID chip 810 of the liquid syringe 800.

At the same time as the liquid condition data is thus displayed on the touch panel 403 and the head display 415, an operating icon for instructing whether to use the liquid condition data as the injection control data is also displayed.

In the case where the operator is to use the liquid condition data displayed as the injection control data, the operator touches the operating icon indicating “accept”. The liquid injector 400 decides, upon detecting such input (step S18), whether the patient has renal dysfunction based on the personal condition data acquired from the imaging order data as above (step S19).

In this process, a plurality of conditions of the personal condition data initially contained in the imaging order data is utilized for deciding whether the patient has renal dysfunction. More specifically, the body weight, age, and serum creatinine value of the patient are extracted from the personal condition data, and the estimated value of the glomerular filtration rate is calculated with the formula of:

(140−age)×body weight/72×serum creatinine value.

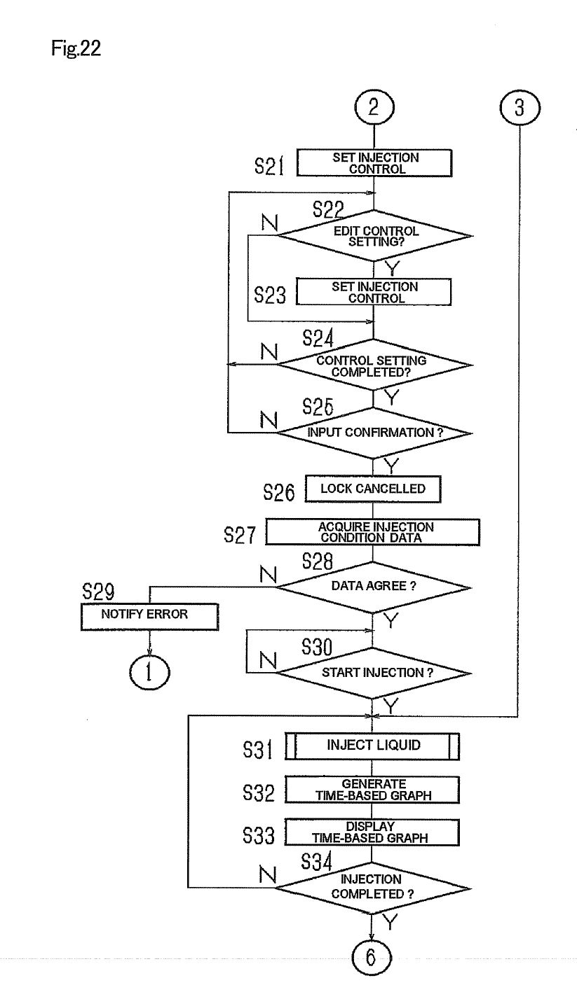

In the case where the estimated value thus calculated is within the predetermined acceptable range, it is decided that the patient does not have renal dysfunction (step S20-N). In this case, as shown in FIGS. 22 and 24, a part of the injection condition data acquired from the imaging order data and a part of the liquid condition data acquired from the liquid syringe 800 are set as a part of the injection control data (step S21).

Then, the product name, lot number, etc., constituting a part of the liquid condition data, are displayed on the touch panel 403 and the head display 415 together with the injection amount and so on constituting a part of the injection control data set based on the liquid condition data.

In the case where the RFID chip 810 is not mounted on the liquid syringe 800, naturally the data is not detected by the RFID reader 416 (step S15-N), and hence the injection control data set based on the injection condition data is utilized (step S21).

With the liquid injector 400 according to this embodiment, even in the case of manually setting the injection control data instead of setting there of based on the injection condition data as stated above (steps S4 to S11), the process of acquiring the liquid condition data from the RFID chip 810 of the liquid syringe 800, thereby setting the liquid condition data as the injection control data as above, remains effective (steps S5 to S8).

The part of the liquid condition data displayed as above includes the product name, expiry, etc. that are useful for the operator to visually confirm. Also, the part of the liquid condition data set as the injection control data includes, naturally, the withstand pressure etc. useful as the injection control data.

In the case where the region to be imaged is registered in the injection condition data while the body weight etc. are not, as shown in FIG. 13, it is only the region to be imaged that is set as the injection control data based on the injection condition data. In this case, the remaining items of the injection control data to be set based on the injection condition data are manually set (steps S22 to S24).

The injection control data set based on the injection condition data and the liquid condition data as above (steps S21 to S24) can also be modified through a manual input by the operator (steps S22, S23).

The liquid injector 400 according to this embodiment is not yet ready to start the liquid injection, at the time of acquiring the injection condition data from the imaging order data as above thereby completing the setting of the injection control data (steps S12 to S24).

Therefore, the operator makes, upon completing the injection control data setting (step S24-Y), the final confirmation on whether the extension tube is free from bubbles and so on, and manually operates the final confirmation switch 414 of the injection head 410, after such confirmation.

The liquid injector 400 decides, upon detecting the input of the final confirmation switch 414 (step S25), that the final confirmation has been executed, and cancels the operation lock against the liquid injection (step S26). However, the liquid injector 400 according to this embodiment is still unable to execute the liquid injection, at the moment that the final confirmation has been executed and the operation lock has been cancelled.

In other words, as shown in FIG. 24, the liquid injector 400 causes the control box 500 to acquire the imaging order data again from the RIS 100 as initially done, upon detecting the input of the final confirmation switch 414 (step S25), and acquires a part of the imaging order data again from the control box 500 as the injection condition data (step S27).

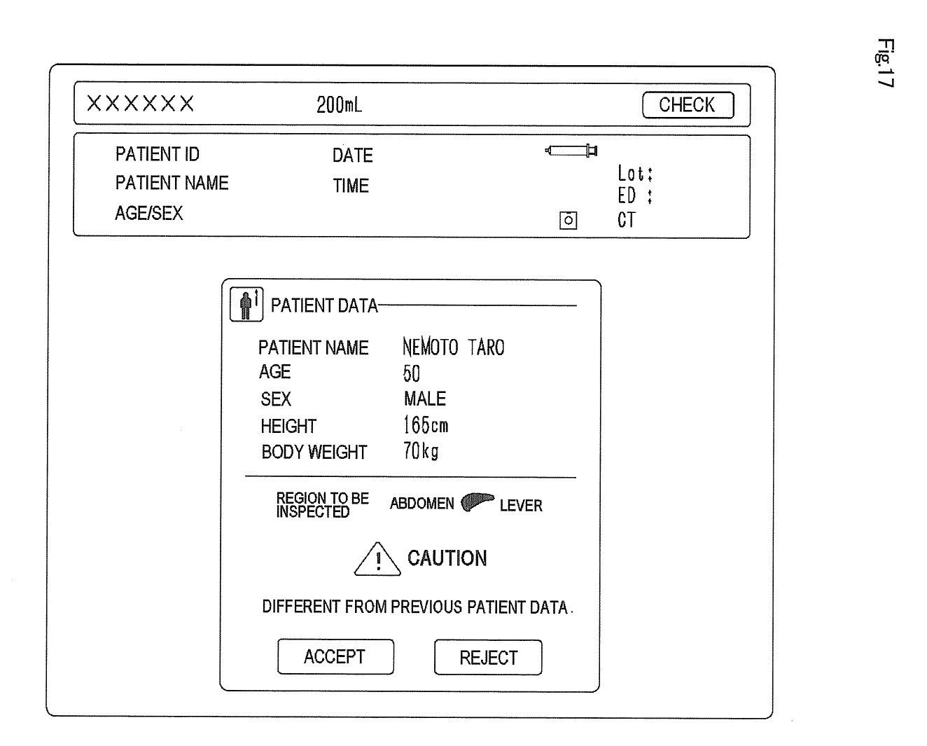

Then the injection condition data first set as the injection control data is compared with the injection condition data again acquired (step S28). In the case where these injection condition data do not agree (step S28-N), an error guidance urging confirmation such as “Different from previous patient data” is displayed on the touch panel 403 and the head display 415, as shown in FIG. 17 (step S29).

In this case, the screen returns to the initial state (step S3), for example by an input of completion of confirmation through the touch panel 403 and the head display 415. Therefore, the injection cannot be started under the modified imaging order data.

On the other hand, once agreement between the injection condition data first set as the injection control data and the injection condition data again acquired is confirmed (step S28-Y), the liquid injector 400 becomes ready to accept the input of the starting instruction through the touch panel 403 and the head display 415.

Once the starting instruction is input (step S30), the syringe driving mechanism 412 is activated under control according to the injection control data that has been set, so that the medical liquid, namely the contrast medium and physiological saline are properly injected to the patient (step S31).

In this process, the lapse of time is measured on a real time basis and the actual injection rate is detected, so that a feedback control is executed upon the syringe driving mechanism 412 such that the injection rate agrees with the injection control data.

Also, the time-based graph indicating the actual injection rate is generated on a real time basis (step S32), and is displayed on the touch panel 403 and the head display 415, for example together with the injection control data (step S33).

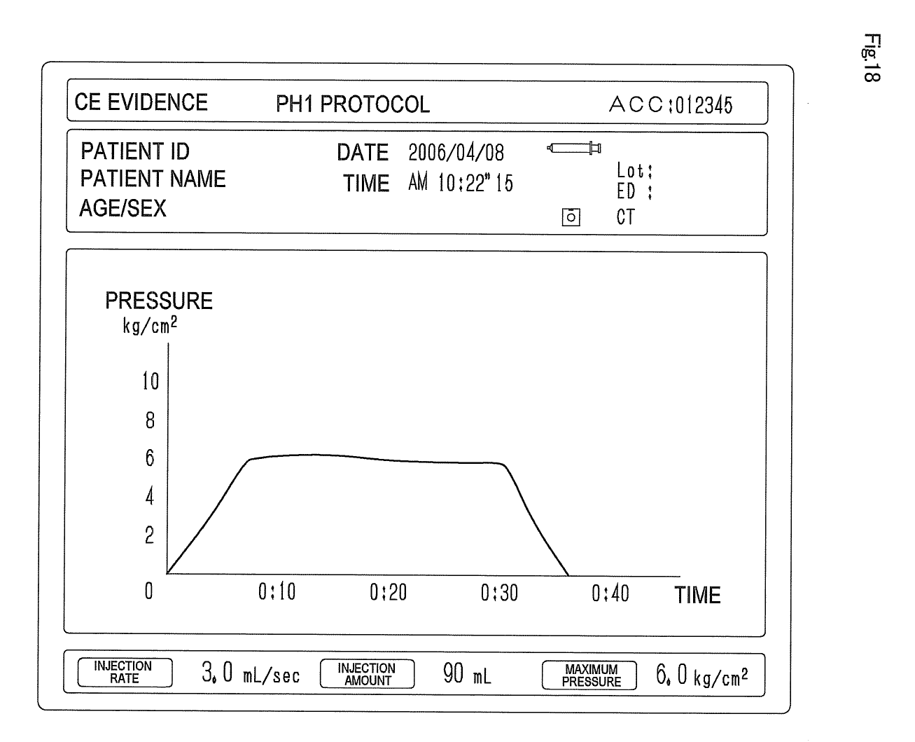

In the case where the injection control data is manually set without acquiring the injection condition data as above, the time-based graph is displayed together with the injection control data manually set, as shown in FIG. 18.

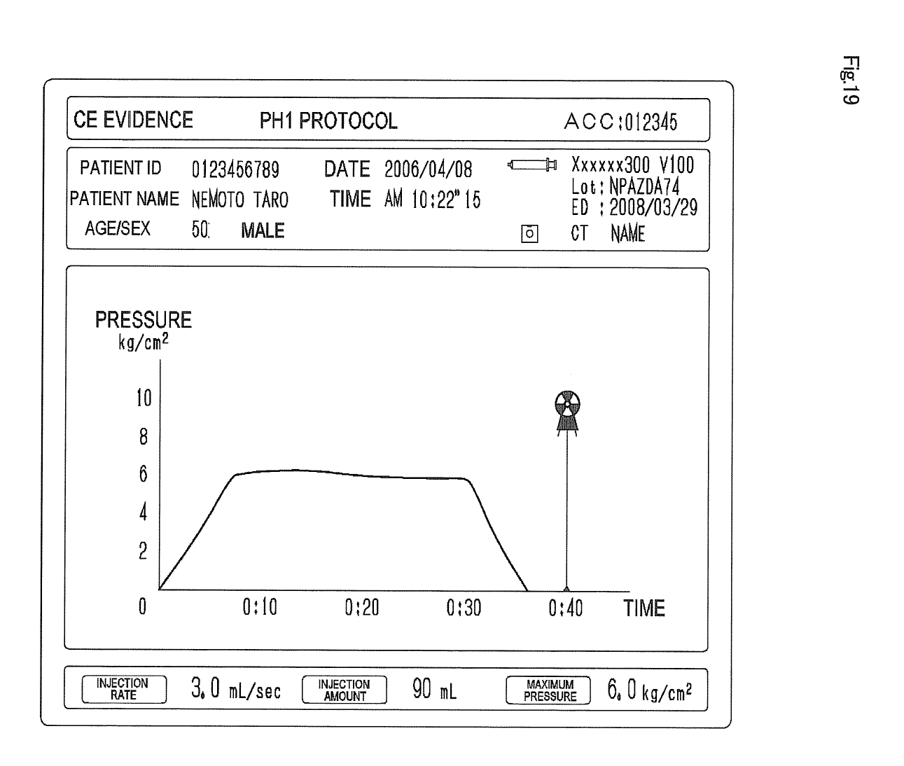

On the other hand, in the case where the injection control data is automatically set upon acquiring the injection condition data and the liquid condition data, the time-based graph is displayed with the injection control data automatically set, and the injection condition data and the liquid condition data, as shown in FIG. 19. In this case, further, a predetermined symbol indicating the starting time of the imaging acquired from the injection condition data is also displayed on the time-based graph.

Here, as shown in FIG. 21, in the case where the estimated value of the glomerular filtration rate, calculated as above based on the body weight, age, and serum creatinine value in the personal condition data contained in the imaging order data, deviates from the acceptable range, the patient is decided to have renal dysfunction (step S20-Y).

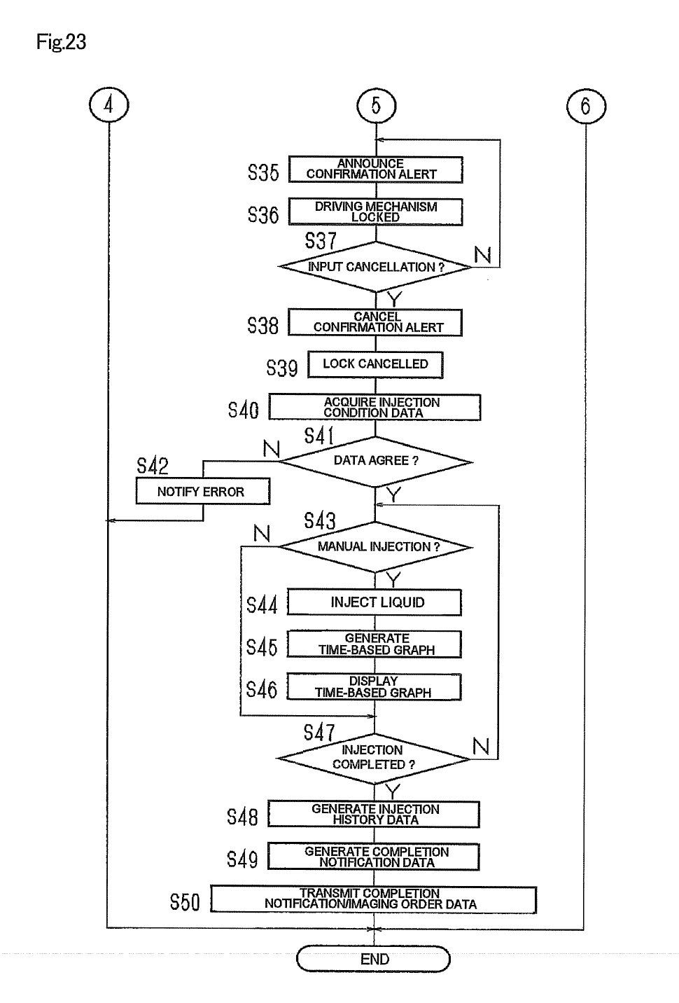

In this case, the guidance data corresponding to such decision is announced as shown in FIG. 23 (step S35), and the syringe driving mechanism 412 is locked so as not to operate (step S36).

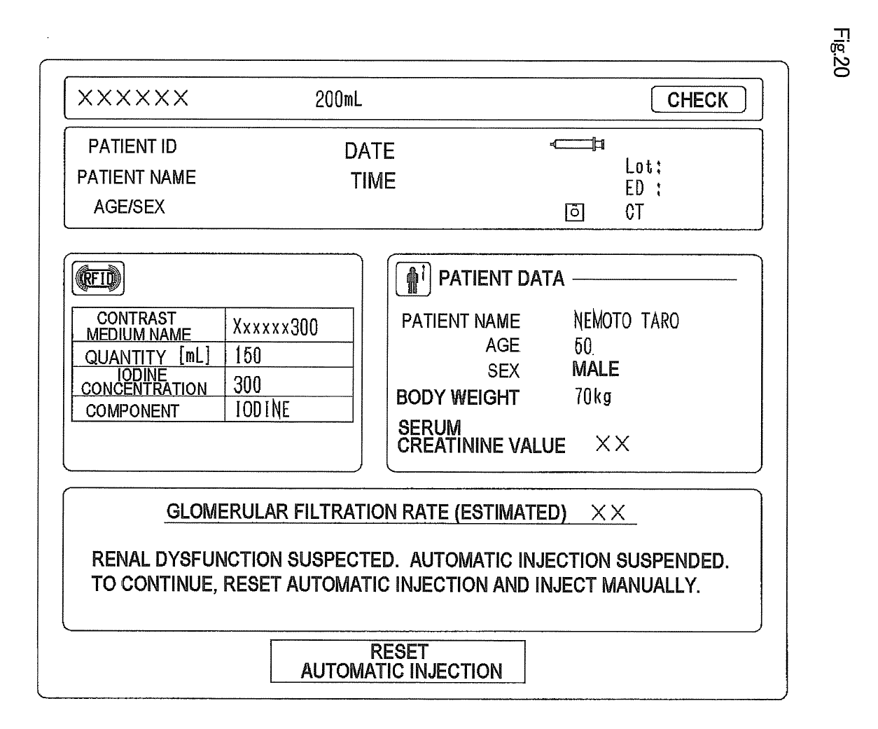

Then the touch panel 403 and the head display 415 display, as shown in FIG. 20, an essential part of the “liquid condition data” such as the product name, and an essential part of the “personal condition data” such as the body weight, which are related to the renal dysfunction.

Also, the guidance data such as “Glomerular filtration rate (estimated value) xx indicates possibility of renal dysfunction. Injection of contrast medium suspended. To inject, reset automatic injection and execute manual injection.” is displayed.

The operator then confirms the estimated value of the glomerular filtration rate to thereby decide whether the contrast medium may be injected. In the case where the operator chooses to manually inject the contrast medium with sufficient care, the operator manually operates the data icon of “reset automatic injection”.

The liquid injector 400 cancels the guidance data (step S38) as well as the operation lock against liquid injection (step S39), upon detecting the resetting operation (step S37-Y).

At this stage, the liquid injector 400 according to this embodiment causes the control box 500 to acquire the imaging order data again from the RIS 100 as initially done, and acquires a part of the imaging order data from the control box 500 again, as the injection condition data (step S40).

Then the personal condition data in the injection condition data set as the injection control data is compared with the personal condition data of the injection condition data acquired again (step S41). In the case where these injection condition data do not agree (step S41-N), an error guidance urging confirmation such as “Different from previous patient data” is displayed on the touch panel 403 and the head display 415, as shown in FIG. 17 (step S42).

In this case, the screen returns to the initial state (step S3), for example by an input of completion of confirmation through the touch panel 403 and the head display 415. Therefore, the injection cannot be started under the modified imaging order data.

On the other hand, once agreement between the personal condition data in the injection condition data first set as the injection control data and the personal condition data in the injection condition data again acquired is confirmed (step S41-Y), the liquid injector 400 becomes ready to execute the liquid injection by manual operation.

At this stage, when the operator manually operates the controller 404 of the liquid injector 400 (step S43-Y), the syringe driving mechanism 412 is activated by that manual operation, so that the liquid such as a contrast medium or physiological saline is injected to the patient (step S44).

Also, the time-based graph indicating the injection rate is generated on a real time basis (step S45), and is displayed on the touch panel 403 and the head display 415, for example together with the injection control data (step S46).

Once the injection job is completed through the automatic operation or manual operation (steps S34-Y, S47-Y), the injection history data including the time-based graph indicating the actual injection rate is generated (step S48).

The injection history data thus generated includes, for example, the image data of the time-based graph and the text data of the injection control data manually set, in the case where the injection control data is manually set without acquiring the injection condition data.

In the case where the injection condition data and the liquid condition data are acquired so that the injection control data is automatically set, and the patient is not decided to have renal dysfunction, the injection history data includes, for example, the image data of the time-based graph, the text data of the injection control data and the injection condition data, and a binary flag indicating that that the patient is not decided to have renal dysfunction.

On the other hand, in the case where the patient is decided to have renal dysfunction and the liquid injection is manually executed, the injection history data includes, for example, the image data of the time-based graph and a binary flag indicating that that the patient is decided to have renal dysfunction.

Here, the foregoing text data includes, for example, the injection job ID which is the exclusive identification data of each injection job, the date and time of the start and finish of the actual injection, the identification data of the liquid injector 400, the injection control data, information on whether all of the injection control data has been manually input, or a part thereof has been acquired from the imaging order data, or a part thereof has been acquired from the liquid condition data, the date and time of the first acquisition and confirmation in the case where the injection control data has been acquired from the imaging order data, and the injection condition data and liquid condition data.

Further, in the case where the liquid condition data is acquired from the RFID chip 810 on the liquid syringe 800, a part of that liquid condition data is also included in the injection history data in a form of text data.

Such text data contains, for example, information on whether the liquid condition data has been acquired from the RFID chip 810 or manually input, the product name of the liquid, the ID, chemical classification, ingredients, and expiry of the liquid, the cylinder withstand pressure, lot number, and price.

The liquid injector 400 also generates, upon completing the injection, completion notification data to which at least the injection job ID is allocated and which serves to notify of the completion (step S49). Upon completing the injection job, therefore, the liquid injector 400 transmits the completion notification data and the injection history data to the control box 500 (step S50).

Then the control box 500 transfers the completion notification data received from the liquid injector 400, to the RIS 100. The RIS 100 stores the completion notification data received, in association with the imaging order data via the injection job ID.

The control box 500 also transfers the injection history data received from the liquid injector 400, to the PACS 300. The PACS 300 stores the received injection history data, utilizing the imaging job ID as the index.

In a normal operation, around the time when the liquid injector 400 completes the injection job as above, the imaging job by the CT scanner 200 is started. In this case, the operator inputs the start of the imaging job to the imaging control unit 210 of the CT scanner 200.

The imaging control unit 210 of the CT scanner 200 then transmits the acquisition request for the imaging order data to the RIS 100. The RIS 100 returns the imaging order data selected as above to the CT scanner 200.

Then the CT scanner 200 controls the action of the fluoroscopic imaging unit 201 according to the imaging order data received by the imaging control unit 210, so that the fluoroscopic image data is picked up.

Once the fluoroscopic imaging unit 201 thus picks up the fluoroscopic image data of the patient, the imaging control unit 210 allocates the fluoroscopic image data with the imaging order data. The imaging control unit 210 then transmits the fluoroscopic image data allocated with the imaging order data to the PACS 300.

The PACS 300 stores the fluoroscopic image data, utilizing the imaging job ID of the imaging order data as the index. When the operator is to review the fluoroscopic image data, the operator may for example manually operate the image viewer 600, to thereby read out the fluoroscopic image data from the PACS 300.

In this case, inputting for example the imaging job ID as the retrieval key causes the fluoroscopic image data corresponding to that imaging job ID to be read out from the PACS 300, and to be displayed on the display unit 602 of the image viewer 600.

At the same time, the injection history data is also read out from the PACS 300 based on the imaging job ID, and the injection history data can also be displayed on the display unit 602 of the image viewer 600, if need be.

From the injection history data thus displayed, it can be confirmed whether the whole injection control data for the injection job has been manually input, or partially acquired from the imaging order data, or partially acquired from the liquid condition data. In the case of acquisition from the imaging order data, the date and time of the first injection and the confirmation, and the acquired injection condition data can also be confirmed.

With the liquid injector 400 according to this embodiment, the action of the syringe driving mechanism 412 is controlled by the injection control unit 421 according to the injection control data set as above, so that the liquid syringe 800 is driven for injecting the liquid loaded in the syringe driving mechanism 412 to the patient.

Here, before the liquid injection is executed, the personal condition data of the patient is acquired by the data input unit 422, and the function decision unit 423 decides whether the patient has renal dysfunction based on the acquired personal condition data.

Then in the case where the patient is not decided to have renal dysfunction the liquid injection is executed without announcing the alert, however in the case where the patient is decided to have renal dysfunction, the alert is announced and the liquid injection is suspended. Such arrangement easily and surely prevents improper injection of the liquid such as a contrast medium to a patient with renal dysfunction.

Besides, the liquid injector 400 acquires the personal condition data containing the serum creatinine value, age, and body weight, and calculates the estimated value of the glomerular filtration rate based on the serum creatinine value, age, and body weight in the acquired personal condition data, to thereby decide that the patient has renal dysfunction in the case where the estimated value thus calculated deviates from the predetermined acceptable range.

Although it is not a common practice that the glomerular filtration rate is registered in the electronic chart, the serum creatinine value is normally registered therein. Accordingly, the liquid injector 400 according to this embodiment can easily and surely decide whether the patient has renal dysfunction based on the electronic chart popularly utilized.

Also, the liquid injector 400 according to this embodiment calculates the estimated value of the glomerular filtration rate with the formula of:

(140−age)×body weight/72×serum creatinine value.

Such arrangement allows properly calculating the estimated value of the glomerular filtration rate through a simple arithmetic process.

The liquid injector 400 according to this embodiment also accepts manual operation for executing the injecting if need be, even when the patient is decided to have renal dysfunction and the syringe driving mechanism 412 is disabled from executing the injecting. Such arrangement allows executing the liquid injection by manual operation based on the judgment of the physician within safe limits, despite the decision that the patient has renal dysfunction made by the liquid injector 400.

Further, the liquid injector 400 according to this embodiment acquires the imaging order data of each imaging job containing the personal condition data and managed by the RIS 100, and decides whether the patient has renal dysfunction based on the personal condition data in the acquired imaging order data. Such arrangement eliminates the need for the operator to input the personal condition data to the liquid injector 400, thereby facilitating automatically acquiring the personal condition data of the patient who is to undergo the fluoroscopic image pickup.

At least a part of the acquired imaging order data is also set as at least a part of the injection control data. Accordingly, the acquired imaging order data is utilized not only for the decision of renal dysfunction but also for controlling the liquid injection. Such arrangement allows easily and properly executing the liquid injection.

Besides, the injection condition data and the liquid condition data automatically acquired are also displayed as above. Accordingly, the operator can easily and surely confirm whether the injection condition data and the liquid condition data are appropriate.

Moreover, the name and sex of the patient as part of the injection condition data, and product name as part of the liquid condition data are also displayed. Such arrangement enables the operator to easily and surely confirm the agreement between the injection condition data and the actual patient, and whether the liquid to be used is appropriate.

The acquisition of the injection condition data including the patient ID and so forth is executed through manipulation of the exclusive icon composed of such a logo as “i” and a human body icon. Such arrangement enables the operator to intuitively execute the acquisition of the injection condition data.

Also, the liquid condition data acquired from the RFID chip 810 is displayed with the predetermined “RFID” logo mark. Such arrangement enables the operator to intuitively confirm that the liquid condition data displayed has been acquired from the RFID chip 810.

Also, the foregoing display also appears on the head display 415 of the injection head 410, in addition to the touch panel 403 on the injection control unit 401 of the liquid injector 400. Therefore, the operator can confirm the injection condition data and the liquid condition data even while working close to the injection head 410.

Further, in the fluoroscopic imaging system 1000 according to this embodiment, the RFID chip 810 of the liquid syringe 800 contains the liquid condition data, and the liquid injector 400 acquires the liquid condition data from the RFID chip 810 through the RFID reader 416. Therefore, the liquid injector 400 can easily and surely acquire the liquid condition data of a large volume from the liquid syringe 800.

In particular, the liquid injector 400 cannot acquire the liquid condition data unless the liquid syringe 800 is properly loaded. Such arrangement allows preventing improper execution of the liquid injection without the liquid syringe 800 properly loaded.

Also, the liquid injector 400 according to this embodiment acquires the injection condition data again from the RIS 100 as a part of the imaging order data once the final confirmation is input right before the injection, and checks the agreement between the injection condition data initially acquired and the injection condition data newly acquired.

The liquid injector 400 does not execute the liquid injection based on the injection control data unless such agreement is confirmed, and therefore the injection according to inappropriate injection control data can be easily and surely prevented.

For example, in the case where the imaging order data has been modified or deleted because of a sudden change of the image pickup schedule, the injection based on the first imaging order data is inhibited from being executed.

Also, since the liquid injector 400 notifies the operator to the effect that the imaging order data is not in accordance, the operator does not fail to recognize and confirm the modification of the imaging order data.

However, in the case where the entire injection control data is manually input, the injection can be started upon completing the setting of the injection control data. Such arrangement allows properly controlling the inhibition and permission of the injecting action, through a simple index.

In a normal injection job utilizing the liquid injector 400, the injection control data is set through the injection control unit 401 located away from the injection head 410 as above, and the operator finally confirms, upon completing the setting, the condition of the liquid syringe 800 and the patient at the position close to the injection head 410.

Here, in the liquid injector 400 according to this embodiment, the final confirmation switch 414 for acquiring the imaging order data again for confirmation the agreement right before starting the injection is provided on the injection head 410.

Therefore, both the reacquisition of the imaging order data and the confirmation of the agreement thereof can be executed, upon executing the final confirmation at the position close to the injection head 410, which is anyway mandatory in the conventional system. In particular, the liquid injector 400 is kept from cancelling the operation lock against the liquid injection unless the final confirmation is executed and the final confirmation switch 414 is operated.

Accordingly, both the cancellation of the lock against the injection action in response to the completion of the final confirmation, and the reconfirmation of the imaging order data can be executed by simply operating the final confirmation switch 414.

Also, in the liquid injector 400, the time-based graph is displayed on a real time basis during the injection according to the injection control data. Such arrangement allows the operator to confirm the injection status on a real time basis.

In the case where the injection control data is acquired from the PACS 300 and set, in particular, the details of the data are displayed with the time-based graph. Such arrangement allows the operator to constantly confirm the details of the injection control data.

Further, in the liquid injector 400 according to this embodiment, in the case where the imaging order data is acquired from the RIS 100, the starting time of the imaging job is displayed on the time-based graph according to the imaging order data.

Accordingly, the operator can confirm the relationship between the progress of the injection and the injection starting time on a real time basis. Besides, since an exclusive logo mark is used to indicate the injection starting time, the operator can intuitively confirm the status.

Further, in the fluoroscopic imaging system 1000 according to this embodiment, the injection history data is also stored in association with the fluoroscopic image data stored in the PACS 300, as already stated. The injection history data also contains the decision whether the patient has renal dysfunction.

Accordingly, at the time of viewing the fluoroscopic image data, it can also be confirmed whether the patient has renal dysfunction, and whether the liquid injection has been manually executed based on that decision. Such arrangement enables the operator viewing the fluoroscopic image data to also confirm, along with the action made by the operator, that the liquid has not been improperly injected to the patient with renal dysfunction.

The injection history data referred to above also contains a part of the injection control data and of the liquid condition data. Accordingly, for example the injection history data, the injection control data, and the liquid condition data can be confirmed upon viewing the fluoroscopic image data. Therefore, it can also be confirmed how the operator viewing the fluoroscopic image data executed the liquid injection for picking up the relevant fluoroscopic image data.

Besides, whereas the fluoroscopic image data and the injection history data are mutually associated via the imaging job ID as stated above, the imaging job ID is acquired by the liquid injector 400 as the imaging order data, when automatically setting the injection control data.

In other words, the imaging order data acquired by the liquid injector 400 from the RIS 100 through the control box 500 can be utilized for both the acquisition of the injection control data and generation of the injection history data.

Also, in the case where the injection job of the contrast medium turns to be suspicious, the injection history data and the fluoroscopic image data can also be confirmed together with the decision on renal dysfunction, the injection control data, and the liquid condition data. Accordingly, the injection history data, the decision on renal dysfunction, the injection control data, and the liquid condition data can be employed as the evidence.

Besides, the units 100 to 600 and 900 of the fluoroscopic imaging system 1000 according to this embodiment mutually execute the data communication in accordance with the DICOM standards. Since it is difficult to falsify the communication data according to the DICOM standards, the injection history data, the injection control data, and the liquid condition data have high admissibility as evidence.

Moreover, the completion notification data of the injection job is transmitted from the liquid injector 400 through the control box 500 to the RIS 100, to be stored therein. Since the RIS 100 stores the completion notification data in association with the imaging order data, the RIS 100 can notify the CT scanner 200, for example, of the time of the start and finish of the liquid injection, together with the imaging order data.

In this case, the operator engaged with the CT scanner 200 can refer to the time of the start and finish of the liquid injection, and hence the operator can adjust the starting time of the image pickup according to the injection time.

It is to be noted that the present invention is in no way limited to the foregoing embodiment, but allows various modifications within the scope of the present invention. To cite some examples, the embodiment exemplifies that the liquid injector 400 acquires the personal condition data containing the serum creatinine value, age, and body weight to thereby calculate the estimated value of the glomerular filtration rate with the formula of:

(140−age)×body weight/72×serum creatinine value.

However, the liquid injector 400 may acquire the personal condition data containing the serum creatinine value, age, body weight and sex, and calculate the estimated value of the glomerular filtration rate of a male patient with the formula of:

(140−age)−body weight/72×serum creatinine value,

and the estimated value of the glomerular filtration rate of a female patient with a formula of:

(140−age)×body weight/72×serum creatinine value×0.85.

Alternatively, the liquid injector 400 may acquire the personal condition data containing the glomerular filtration rate, so that the function decision unit 423 may decide that the patient has renal dysfunction in the case where the glomerular filtration rate in the acquired personal condition data deviates from the predetermined acceptable range.

Further, the liquid injector 400 may acquire the personal condition data containing the serum creatinine value, to thereby decide that the patient has renal dysfunction in the case where the serum creatinine value in the acquired personal condition data deviates from the predetermined acceptable range.

The embodiment exemplifies that the syringe driving mechanism 412 is disabled from working in the case where the patient is decided to have renal dysfunction. However, the injecting action of the syringe driving mechanism 412 may be adjusted, in the case where the patient is decided to have renal dysfunction.

In the latter case, the injecting action of the syringe driving mechanism 412 may be adjusted based on the serum creatinine value acquired as above, or based on the glomerular filtration rate acquired as above, or based on the estimated value of the glomerular filtration rate calculated as above.

Such adjustment of the injecting action may be executed, for example, through controlling the syringe driving mechanism 412 so as to adjust at least one of the injection rate, injection duration, and the total injection amount. To be more detailed, the adjustment methods may include lowering the injection rate, shortening the injection duration, and decreasing the total injection amount, according to the extent of the renal dysfunction.

Alternatively, to adjust the injecting action, the injection ratio of the contrast medium and the physiological saline may be modified. For example, the total injection amount of the contrast medium may be decreased while increasing the total injection amount of the physiological saline, according to the extent of the renal dysfunction.

Also, the embodiment only exemplifies that the liquid condition data acquired from the liquid syringe 800 is utilized as a part of the injection control data. However, such liquid condition data may be utilized for controlling the injection according to the extent of the renal dysfunction as above. In this case, for example, the total injection amount may be adjusted based on the concentration of the contrast medium, in the case where the patient is decided to have renal dysfunction.

The embodiment exemplifies that the liquid injector 400 stores the decision result on the renal dysfunction in the PACS 300 together with the injection history data. However, the liquid injector 400 may return the decision result on the renal dysfunction to the RIS 100 or HIS 900, so that either of those units stores that decision together with the imaging order data and the electronic chart.

The embodiment exemplifies that the RFID chip 810 on the liquid syringe 800 contains the liquid condition data, and the liquid injector 400 acquires the liquid condition data through the RFID reader 416.

However, the liquid condition data may be recorded on the liquid syringe in a form of a magnetic stripe, a two-dimensional code, or a barcode, so that the liquid injector acquire such liquid condition data with a magnetic head, a two-dimensional scanner or a line scanner (not shown).

The embodiment exemplifies that the liquid condition data recorded on the liquid syringe 800 includes various contents. However, the liquid condition data recorded on the liquid syringe may solely include the liquid ID.

In this case, the various contents in the liquid condition data may be recorded in a database of the HIS 900, the RIS 100, or the PACS 300 together with the liquid ID, and such various contents of the liquid condition data may be retrieved with the liquid ID as the index, to be thereby acquired by the liquid injector (not shown).

In the foregoing embodiment, it is assumed that the liquid syringe 800 is a prefilled syringe, and the liquid condition data is recorded by the syringe manufacturer. However, the liquid syringe may be a refill syringe and the liquid condition data may be recorded at the medical site according to the liquid to be loaded.

The embodiment exemplifies that the liquid injector 400 automatically sets the injection control data based on the imaging order data acquired from the RIS 100 and the liquid condition data acquired from the liquid syringe 800.

However, the injection control data may be registered in the PACS 300 for example, together with the injection history data and the patient ID, so that such injection control data may be acquired by the liquid injector 400 from the PACS 300 with the patient ID as the index, to be utilized for the injection job.

In this case, in the case where the same patient has undergone the imaging job in the past, the preceding injection control data may be reutilized. Accordingly, even though complicated injecting actions are specified in the injection control data, such injecting action can be easily reproduced.

Also, the injection history data generated based on the injection job in the liquid injector 400 may be registered in the PACS 300 with the patient ID for example, and such injection history data may be acquired by the liquid injector 400 from the PACS 300 with the patient ID as the index, to be utilized for the injection job as the injection control data.

In this case, although the injection control data has to be generated from the injection history data in the liquid injector 400, registering the injection history data in the PACS 300 allows skipping the registration of the injection control data.

Also, the preceding injection control data acquired by the liquid injector 400 from the PACS 300 may be automatically adjusted according to the new injection condition data. For example, the body weight of the patient, the concentration and ingredients of the liquid, and so forth may be utilized as such injection condition data.

For example, in the case where the patient's body weight allocated to the preceding injection control data acquired from the PACS 300 by the liquid injector 400 is 50 kgs., while the current body weight input in the liquid injector 400 is 60 kgs., it is preferable to increase the injection rate and total injection amount in the injection control data by 20% (=60/50).

Likewise, in the case where the component concentration of the liquid allocated to the preceding injection control data is 10%, while the current component concentration input in the liquid injector 400 is 20%, it is preferable to decrease the injection rate and total injection amount in the injection control data to 50% (−10/20).

The foregoing embodiment also exemplifies that the acquisition of the patient ID and the region to be imaged by the liquid injector 400 from the imaging order data registered in the RIS 100 exempts the operator from inputting the patient ID and so on to the liquid injector 400, thereby preventing an erroneous input.

However, a part or all of the patient ID, the region to be imaged and so forth may be input to the liquid injector 400. Also, the fluoroscopic imaging system 1000 may include a patient management medium (not shown) with respect to each patient, in which an RFID chip containing at least the patient ID is mounted, so that the liquid injector 400 may acquire the patient ID from the RFID chip in the patient management medium.

Such patient management medium may be realized in a form of, for example, an electronic chart with the RFID chip mounted thereon, or a managing arm band to be attached to the patient's arm (not shown).

Such arrangement also exempts the operator from inputting the patient ID, thereby preventing an erroneous input. Further, the personal condition data may be registered in the RFID chip of the patient management medium, so that the liquid injector 400 may acquire such data and utilize for the automatic setting of the injection control data and decision on renal dysfunction.

The foregoing embodiment exemplifies that the acquisition of the injection condition data in the imaging order data in the RIS 100 by the liquid injector 400 again right before the injection enables coping with the modification of the imaging order data.

However, liquid injector 400 may also confirm the agreement between the patient ID acquired from the imaging order data in the RIS 100 and the patient ID acquired from the RFID chip in the patient management medium, so as to inhibit the action control of the syringe driving mechanism 412 until the agreement is confirmed, and to output a predetermined alert for reconfirmation in the case of disagreement.

The embodiment only exemplifies that the liquid injector 400 utilizes the liquid condition data acquired from the liquid syringe 800 for setting the injection control data. However, the liquid injector 400 may transmit the liquid condition data acquired from the liquid syringe 800 to the HIS 900, as stated above.

In general, the HIS 900 executes the accounting process with respect to each imaging job. Accordingly, in the case where the liquid condition data of the liquid actually utilized for the injection is acquired, the HIS 900 can easily and quickly execute the accounting process.

The embodiment exemplifies that in the case where a plurality of injection control data corresponding to the patient ID provided by the liquid injector 400 is registered in the PACS 300, the plurality of injection control data is transmitted from the PACS 300 to the liquid injector 400, so that the liquid injector 400 selects the latest one of the injection control data.

However, the PACS 300 may select the latest one of the injection control data and transmit such data to the liquid injector 400. In this case, although the PACS 300 is required to have an additional exclusive process, the amount of data to be transmitted can be reduced, so as to prevent congestion.

Also, the plurality of injection control data transmitted from the PACS 300 may be displayed on the liquid injector 400 in a form of a listing, so that one of the listed injection control data is selected. In this case, the operator can select the desired optimal injection control data. Further, the liquid injector 400 may select one out of the plurality of injection control data transmitted from the PACS 300 according to a predetermined condition, for example that the body weight is closest.

The embodiment exemplifies that the entirety of the imaging order data managed by the RIS 100 is acquired by the control box 500, and the liquid injector 400 acquires a part of the imaging order data from the control box 500 as the injection condition data. However, the liquid injector 400 may acquire the entirety of the imaging order data as the injection condition data.

The embodiment exemplifies that the RIS 100 is of the push-type, and the control box 500 acquires the proper imaging order data at a predetermined timing. However, the RIS 100 may be of the pull-type.

In the latter case, the CT scanner 200 transmits the acquisition request for the imaging order data to the RIS 100 with at least an order retrieval key. Then the RIS 100 selects one of the plurality of imaging order data according to the acquisition request and the order retrieval key received from the CT scanner 200, and returns the selected data.

The control box 500 then transmits to the RIS 100 the acquisition request for the imaging order data received from the liquid injector 400. The RIS 100 returns one of the imaging order data selected according to the acquisition request received from the control box 500.

Alternatively, the RIS 100 may return a plurality of imaging order data according to the acquisition request received from the CT scanner 200. In this case, the CT scanner 200 accepts an operation of selecting one of the plurality of imaging order data returned, and notifies the RIS 100 of the selected imaging order data.

The RIS 100 may also retrieve a part of the plurality of imaging order data based on the acquisition request and the order retrieval key received from the CT scanner 200, and return the retrieved data. The CT scanner 200 accepts an operation of selecting one of the imaging order data returned, and notifies the RIS 100 of the selected imaging order data.

Once the control box 500 transmits the acquisition request for the imaging order data to the RIS 100, the RIS 100 returns the one of the imaging order data notified of by the CT scanner 200, according to the acquisition request received from the control box 500.

Such arrangement allows the control box 500 to acquire the proper imaging order data despite that the RIS 100 is of the pull-type, and to allocate the imaging job ID and so on to the injection history data.

The foregoing embodiment exemplifies that the control box 500 acquires the imaging order data from the RIS 100. However, the RIS 100 and the CT scanner 200 may be connected via the control box 500, so that the control box 500 may acquire the imaging order data which is transmitted from the RIS 100 to the CT scanner 200.

Also, the control box 500 may be connected to the CT scanner 200 without being connected to the RIS 100, and may acquire the imaging order data from the CT scanner 200.

In this case, for example, the control box 500 may transfer the acquisition request received from the liquid injector 400 to the CT scanner 200, and the CT scanner 200 may return the imaging order data according to the acquisition request received from the control box 500.

Alternatively, the CT scanner 200 may accept an operation of selecting one of the plurality of imaging order data returned from the pull-type RIS 100, to thereby transfer the selected imaging order data to the control box 500.

Also, the control box 500 may be connected to the RIS 100 and the CT scanner 200, so that the first imaging order data may be acquired from the RIS 100, and the imaging order data for confirmation may be acquired from the CT scanner 200.

The embodiment exemplifies that the injection history data and the injection control data generated in the liquid injector 400 are stored in the PACS 300 together with the fluoroscopic image data generated in the CT scanner 200.

However, the injection history data and the injection control data may be transmitted from the liquid injector 400 to the RIS 100 through the control box 500, so that the RIS 100 may store the injection history data and the injection control data. In this case, the RIS 100 can manage the imaging order data, the injection history data, and the injection control data in mutual association via the job ID or the like.

Even in this case, the fluoroscopic image data registered in the PACS 300 is also allocated with the job ID of the imaging order data, and hence the fluoroscopic image data can be associated with the injection history data and the injection control data.

The embodiment exemplifies that the entirety of the imaging order data is allocated to the fluoroscopic image data, when stored in the PACS 300. However, only the imaging job ID of the imaging order data may be allocated to the fluoroscopic image data.

Even in this case, the fluoroscopic image data can be associated with the injection history data and the injection control data via the imaging job ID, and therefore the imaging order data can be read out from the RIS 100 with the imaging job ID.

Alternatively, only the imaging job ID of the imaging order data may be allocated to the fluoroscopic image data, and the entirety of the imaging order data may be allocated to the injection history data and the injection control data, and also the imaging order data may be divided into portions to be allocated to the fluoroscopic image data, and to each of the injection history data and the injection control data. Also, the entirety of the display image on the touch panel 403 and the head display 415 of the liquid injector 400 may be included in the injection history data.

The foregoing embodiment only exemplifies the case where the injection condition data is set in the liquid injector 400. However, the injection condition data may be notified from the liquid injector 400 to the control box 500, and then from the control box 500 to the RIS 100. In the latter case, the injection condition data may be notified from the RIS 100 to the CT scanner 200, together with the imaging order data.

Such arrangement allows the person operating the CT scanner 200 to refer to the injection condition data, and therefore to adjust the imaging action according to the injection condition data. Further, automatic adjustment of the imaging action can also be executed according to the injection condition data acquired by the imaging control unit 210 of the CT scanner 200.

The embodiment exemplifies that the liquid injector 400 completes the injection history data before transmitting to the control box 500. However, the liquid injector 400 may transmit the injection history data in divided portions to the control box 500, so that the control box 500 integrates the injection history data.

More specifically, the liquid injector 400 may transmit the injection condition data and the starting date and time to the control box 500 upon starting the injection, the injection rate and so on time after time during the injection, and the finishing date and time upon completing the injection. In this case, the control box 500 can complete the injection history data from various data accumulated during the period from the start of the injection to the finish thereof.

The embodiment exemplifies that the respective units 100 to 600 and 900 mutually perform the data communication according to DICOM standard which is difficult to falsify, thereby securing high admissibility of the injection history data as evidence. However, the liquid injector 400 may generate the injection history data in another data format that is difficult to falsify, such as the Portable Document Format (PDF).

Likewise, the control box 500 may convert the injection history data received from the liquid injector 400 in the Joint Photographic Coding Experts Group (JPEG) format into the PDF format. Further, the liquid injector 400 and the control box 500 may be connected to what is known as the Internet, so as to acquire an electronic signature and allocate the injection history data with the same.

The embodiment exemplifies that the head display 415 is directly attached to the injection head 410 so as to extend downward from a rear portion of the left side thereof, which is closer to the operator. However, the head display 415 may be attached to any position as long as the operation of the injection head 410 is not disturbed and the screen display can be confirmed.

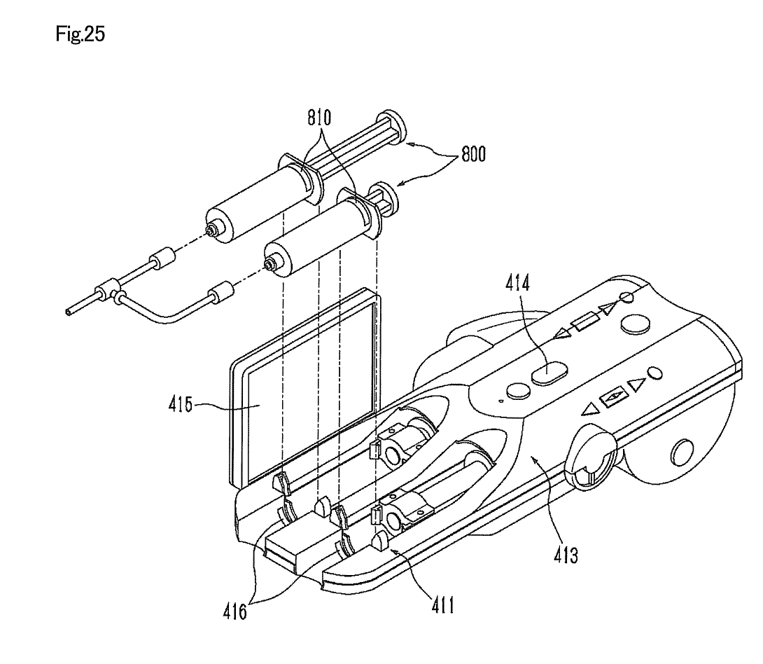

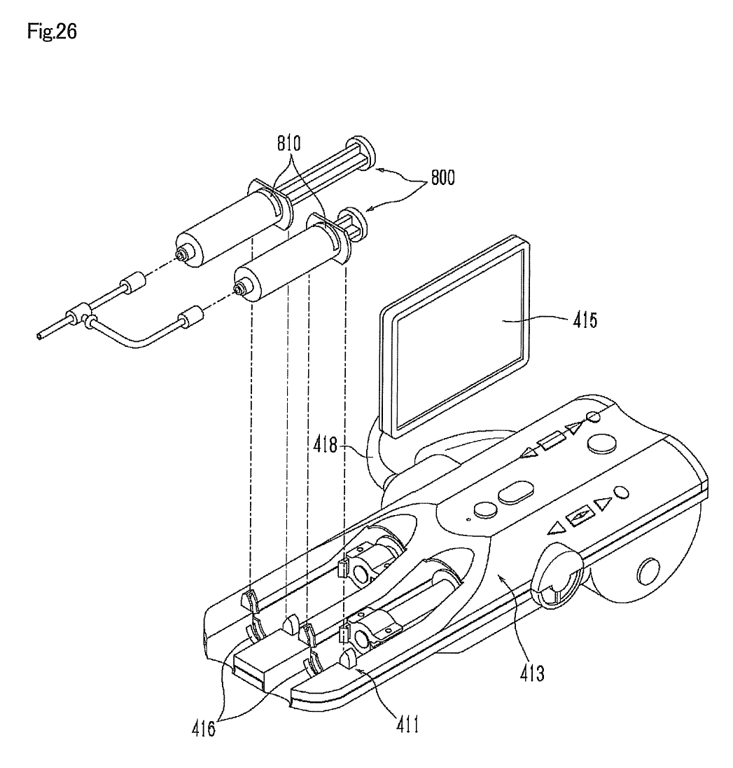

For example, the head display 415 may be attached to the right side or a forward portion of the injection head 410, or so as to extend upward therefrom, as shown in FIG. 25. Also, as shown in FIG. 26, the head display 415 may be pivotably mounted on the injection head 410 via a movable arm 418 or the like.

The embodiment exemplifies that the liquid injector 400 utilizes a pair of liquid syringes 800 to inject the contrast medium and physiological saline to the patient. However, the liquid injector may utilize a single liquid syringe 800 to inject the contrast medium and physiological saline to the patient (not shown).

The embodiment exemplifies that the CT scanner 200 serves as the imaging diagnostic apparatus, and the liquid injector 400 injects the contrast medium for CT scanning as the medical liquid. However, the imaging diagnostic apparatus may be constituted of a MRI equipment, a PET equipment, or an ultrasonic diagnostic equipment, and the liquid injector may inject the contrast medium prepared exclusively for such equipments.

The embodiment exemplifies that the CT scanner 200 and the liquid injector 400 are independently activated on a stand-alone basis. However, the CT scanner 200 and the liquid injector 400 may work in correlation to perform various actions, through data communication.

The embodiment exemplifies that the fluoroscopic imaging system 1000 includes one each of the respective units, for the sake of explicitness of the description. However, in a large-scale hospital or the like, each of a plurality of fluoroscopic imaging systems may include one each of the RIS 100, the CT scanner 200, the liquid injector 400, and the control box 500, and the plurality of fluoroscopic imaging systems may share the PACS 300 and the image viewer 600 (not shown). In such case also, the hardware such as the RIS 100, the PACS 300, and the image viewer 600 may be prepared in a plurality of numbers and connected in parallel (not shown).

Further, the embodiment exemplifies that the fluoroscopic image data, the injection history data, and the injection control data are stored in a single unit of the PACS 300. However, the hardware that stores the fluoroscopic image data, the injection history data, and the injection control data may be independently prepared and connected via the communication network.

Further, the embodiment exemplifies that the RIS 100, the CT scanner 200, the PACS 300, the liquid injector 400, the control box 500, the image viewer 600, and the HIS 900 are separately constructed and mutually connected via the communication network 700 to 706.

However, the respective units 100 to 600 and 900 may be integrally constructed in various combinations. To cite a few examples, the injection control unit 401 of the liquid injector 400 and the control box 500 may be integrally constituted; the RIS 100 and the PACS 300 may be added to such combination to thereby form a unified structure; and the PACS 300 and the image viewer 600 may be integrally constituted.

Also, the control box 500 may be unified with the RIS 100 and the PACS 300, and the control box 500, the PACS 30, and the image viewer 600 may be integrally constituted.

Also, the imaging control unit 210 of the CT scanner 200, the RIS 100, and the control box 500 may be integrally constituted; the imaging control unit 210 of the CT scanner 200, the PACS 300, and the control box 500 may be integrally constituted; and the image viewer 600 may be added to thereby form a unified structure.

Further, the image viewer 600 and the PACS 300 may be integrally constituted, and the control box 500 and the imaging control unit 210 of the CT scanner 200 may be added to thereby form a unified structure.

Still further, the embodiment exemplifies that the computer unit works according to the computer program, to thereby logically realize the respective units 100 to 600 and 900 to perform the assigned functions.

However, it is also possible to set up the respective units as individually independent hardware, or some units as hardware and the others as software.

Naturally, the foregoing embodiment and the plurality of variations may be combined, unless contradiction arises. Further, although the foregoing embodiment and variations represent the specific structure of the respective constituents, such structure may be modified in various manners provided that the intended function according to the present invention is satisfied.

1. A liquid injector that injects a medical liquid to a patient whose fluoroscopic image data is to be picked up, comprising:
a liquid injection mechanism that executes injection of said medical liquid;
an injection control unit that controls an action of said liquid injection mechanism according to injection control data to be given;
a data input unit that acquires personal condition data originating from said patient before executing said liquid injection; and
a function decision unit that decides renal dysfunction of said patient based on said personal condition data acquired.

2. The liquid injector according to claim 1,
wherein said data input unit acquires said personal condition data containing a serum creatinine value; and
said function decision unit decide that said patient has renal dysfunction in the case where said serum creatinine value in acquired said personal condition data deviates from a predetermined acceptable range.

3. The liquid injector according to claim 1,
wherein said data input unit acquires said personal condition data containing a glomerular filtration rate; and
said function decision unit decides that said patient has renal dysfunction in the case where said glomerular filtration rate in said acquired personal condition data deviates from a predetermined acceptable range.

4. The liquid injector according to claim 1,
wherein said data input unit acquires said personal condition data containing said serum creatinine value, age and body weight; and
said function decision unit calculates an estimated value of said glomerular filtration rate based on said serum creatinine value, age and body weight in said acquired personal condition data, and decide that said patient has renal dysfunction in the case where said estimated value thus acquired deviates from a predetermined acceptable range.

5. The liquid injector according to claim 4,
wherein said function decision unit calculates said estimated value of said glomerular filtration rate through a formula of:
(140−age)×body weight/72×serum creatinine value.

6. The liquid injector according to claim 1,
wherein said data input unit acquires said personal condition data containing said serum creatinine value, age, body weight, and sex; and
said function decision unit calculates an estimated value of said glomerular filtration rate based on said serum creatinine value, age, body weight, and sex in said acquired personal condition data, and decide that said patient has renal dysfunction in the case where said estimated value thus acquired deviates from a predetermined acceptable range.

7. The liquid injector according to claim 6,
wherein said function decision unit calculates said estimated value of said glomerular filtration rate through a formula of:
(140−age)×body weight/72×serum creatinine value
in the where said sex is male, and through a formula of:
(140−age)×body weight/72×serum creatinine value×0.85
in the case where said sex is female.

8. The liquid injector according to claim 1, further comprising an alert notification unit that outputs a confirmation alert in the case where said patient is decided to have renal dysfunction.

9. The liquid injector according to claim 1,
wherein said injection control unit disables said liquid injection mechanism from working in the case where said patient is decided to have renal dysfunction.

10. The liquid injector according to claim 1,
wherein said injection control unit adjusts said injecting action of said liquid injection mechanism in the case where said patient is decided to have renal dysfunction.

11. The liquid injector according to claim 2,
wherein said injection control unit adjusts said injecting action of said liquid injection mechanism according to said acquired serum creatinine value.

12. The liquid injector according to claim 3,
wherein said injection control unit adjusts said injecting action of said liquid injection mechanism according to said acquired glomerular filtration rate.

13. The liquid injector according to claim 4,
wherein said injection control unit adjusts said injecting action of said liquid injection mechanism according to said calculated estimated value of said glomerular filtration rate.

14. The liquid injector according to claim 10,
wherein said injection control unit adjusts at least one of injection rate, injection duration, and a total injection amount provided by said liquid injection mechanism.

15. The liquid injector according to claim 10,
wherein said liquid injection mechanism injects a contrast medium and physiological saline as medical liquid; and
said injection control unit adjusts an injection ratio of said contrast medium and said physiological saline.

16. The liquid injector according to claim 10, further comprising a liquid acquisition unit that acquires liquid condition data originating from said medical liquid;
wherein said injection control unit adjusts said injecting action according to said liquid condition data in the case where said patient is decided to have renal dysfunction.

17. The liquid injector according to claim 16,
wherein said liquid injection mechanism drives a liquid syringe bearing said liquid condition data and loaded with said medical liquid; and
said liquid acquisition unit acquires said liquid condition data from said liquid syringe.

18. The liquid injector according to claim 17,
wherein said liquid injection mechanism drives a liquid syringe on which an RFID chip containing said liquid condition data is mounted; and
said liquid acquisition unit acquires said liquid condition data from said RFID chip.

19. The liquid injector according to claim 16,
wherein said injection control unit further comprises a control setting unit that sets at least a part of said acquired liquid condition data in said injection control unit as at least a part of said injection control data.

20. The liquid injector according to claim 1,
wherein said data input unit acquires imaging order data of each imaging job containing said personal condition data and managed outside; and
said function decision unit decides whether said patient has renal dysfunction based on said personal condition data in said acquired imaging order data.

21. The liquid injector according to claim 20, further comprising a control setting unit that sets at least a part of said acquired imaging order data in said injection control unit as at least a part of said injection control data.

22. The liquid injector according to claim 20, further comprising a data output unit that transmits a decision result of renal dysfunction to outside, to thereby store said decision result outside together with said imaging order data.

23. The liquid injector according to claim 1, further comprising:
a history generation unit that generates injection history data containing action history of said liquid injection mechanism based on said injection control data; and
a data output unit that transmits said generated injection history data to outside, to thereby store said injection history data outside together with said fluoroscopic image data.

24. The liquid injector according to claim 23,
wherein said history generation unit also registers at least a part of said injection control data in said injection history data.

25. The liquid injector according to claim 23,
wherein said history generation unit also registers said decision result of renal dysfunction in said injection history data.

26. A fluoroscopic imaging system comprising an external processor that contains imaging order data relevant to each imaging job of picking up fluoroscopic image data from a patient, and a liquid injector that injects a medical liquid to said patient;
wherein said external processor contains said imaging order data including personal condition data originating from said patient; and
said liquid injector is one according to claim 20.

27. A fluoroscopic imaging system comprising a liquid injector that injects a medical liquid to a patient whose fluoroscopic image data is to be picked up, and a data storage unit that stores therein said fluoroscopic image data picked up;
wherein said liquid injector is one according to claim 23; and
said data storage unit also stores injection history data received from said liquid injector, together with said fluoroscopic image data.

28. A computer program for a liquid injector that includes a liquid injection mechanism for injecting a medical liquid to a patient whose fluoroscopic image data is to be picked up, comprising causing said liquid injector to execute:
an injection control process including controlling an action of said liquid injection mechanism according to injection control data to be set;
a data input process including acquiring personal condition data originating from said patient; and
a function decision process including deciding whether said patient has renal dysfunction based on said personal condition data acquired.
